(19) United States
(12) Patent Application Publication
(10) Pub. No.: US 2016/0017035 A1
(43) Pub. Date: Jan. 21, 2016

(54) TIM RECEPTORS AS VIRUS ENTRY COFACTORS

(71) Applicant: INSTITUT NATIONAL DE LA SANTE ET DE LA RECHERCHE MEDICALE (INSERM), Paris (FR)

(72) Inventors: Ali Amara, Paris (FR); Laurent Meertens, Paris (FR)

(73) Assignee: INSTITUT NATIONAL DE LA SANTE ET DE LA RECHERCHE MEDICALE (INSERM), Paris (FR)

(21) Appl. No.: 14/379,879

(22) PCT Filed: Feb. 20, 2013
(86) PCT No.: PCT/EP13/53391

§ 371 Date: Aug. 20, 2014

(30) Foreign Application Priority Data
Feb. 21, 2012 (EP) 12305193.0
Oct. 17, 2012 (EP) 12306281.2

(51) Int. Cl. C07K 16/28; C07K 14/705; A61K 38/17; A61K 31/713; A61K 45/06; A61K 39/395; C12N 15/113; C07K 16/44

(52) U.S. Cl. CPC C07K 16/2803; C12N 15/1138; C07K 14/70503; C07K 16/44; A61K 31/713; A61K 45/06; A61K 39/3955; A61K 39/39583; A61K 38/1774; C12N 2310/111; C12N 2310/14; C07K 2317/76

(57) ABSTRACT

The present invention concerns the use of an inhibitor of an interaction between phosphatidylserine and a TIM receptor for preventing or treating a virus entry cofactors, in particular phosphatidylserine harboring virus infection such as flavivirus infection.

### FIELD OF THE INVENTION

The present invention concerns the use of an inhibitor of an interaction between phosphatidylserine and a TIM receptor for preventing or treating a viral infection.

### BACKGROUND TO THE INVENTION

Viral infections are a major threat to public health. The emergence and expansion of life-threatening diseases caused by viruses (e.g. hemorrhagic fever and encephalitis), together with unmet conventional prevention approaches (e.g., vaccines) highlights the necessity of exploring new strategies that target these deadly pathogens.

The Flavivirus genus for example encompasses over 70 small-enveloped viruses containing a single positive-stranded RNA genome. Several members of this genus such as Dengue virus (DV), Yellow Fever Virus (YFV), and West Nile virus (WNV), are mosquito-borne human pathogens causing a variety of medically relevant human diseases including hemorrhagic fever and encephalitis (Gould and Solomon, 2008, Lancet, 371:200-509; Gubler et al., 2007, Fields Virology, 5^{th} Edition, 1153-1252). Dengue disease, which is caused by four antigenically related serotypes (DV1 to DV4), has emerged as a global health problem during the last decades and is one of the most medically relevant arboviral diseases. It is estimated that 50-100 million dengue cases occur annually and more than 2.5 billion people being at risk of infection. Infection by any of the four serotypes causes diseases, ranging from mild fever to life-threatening dengue hemorrhagic fever (DHF) and dengue shock syndrome (DSS). Despite the importance and increasing incidence of DV as a human pathogen, there is currently no licensed vaccine available against DV and the lack of anti-viral drugs severely restricts therapeutic options.

Future efforts to combat dengue disease require a better understanding of the DV life cycle. DV entry into target cells is a promising target for preventive as well as therapeutic anti-viral strategies since it is a major determinant of the host-range, cellular tropism and viral pathogenesis. During primary infection, DV enters host cells by clathrin-mediated endocytosis, a process driven by the interaction between the viral glycoprotein (E protein) with cellular receptors. Within the endosome, the acidic environment triggers an irreversible trimerization of the E protein that results in fusion of the viral and cell membranes, allowing the release of the viral capsid and genomic RNA into the cytosol. To date, the molecular bases of DV-host interactions leading to virus entry are poorly understood and little is known about the identity of the DV cellular receptor(s). DV is known to infect a wide range of cell types. DV may thus exploit different receptors, depending on the target cell, or use widely expressed entry molecules. Earlier studies indicated that DV virions make initial contact with the host by binding to heparan-sulfate proteoglycans on the cell membrane. These molecules recognize the positively charged residues on the surface of E protein and are thought to concentrate the virus at the target cell surface before its interactions with entry factors. Numerous cellular proteins such as heat shock protein 70 (HSP70), HSP90, GRP78/Bip, a lipopolysaccharide receptor-CD14 or the 37/67 kDa high affinity laminin have been proposed as putative DV entry receptors. However, their function in viral entry remains poorly characterized and of unclear physiological relevance. To date, the only well-characterized factors that actively participate in the DV entry program are DC-SIGN expressed on dendritic cells, L-SIGN expressed on liver sinusoidal endothelial cells and the mannose receptor (MR) expressed on macrophages. These molecules belong to the C-type lectin receptor family and bind mannose-rich N-linked glycans expressed on the DV E protein. However, DV infects cell types that do not express DC-SIGN, MR or L-SIGN, indicating that other relevant entry receptor(s) exist and remain to be identified.

Currently, DV has become a global problem and is endemic in more than 110 countries. Thus, development of a prophylactic or curative treatment DV infection is needed.

Moreover, deciphering the mechanism of DV internalization might also pave the way to developing treatment of other viral infections.

### DESCRIPTION OF THE INVENTION

The inventors have found that DV infection is mediated by the interaction between phosphatidylserine (PtdSer) present at the surface of the DV viral envelope and TIM receptor present at the surface of the host cell, and that such interaction can be blocked, thereby inhibiting entry of DV into host cells and preventing DV infection.

Furthermore, the inventors found that this interaction between phosphatidylserine (PtdSer) and TIM receptors is not only used by other flavivirus such as Yellow Fever Virus (YFN) and West Nile Virus (WNV) but also for example by the Chikungunya Virus showing that this interaction may represent a general mechanism exploited by viruses that incorporate phosphatidylserine (PtdSer) in their membrane.

Thus, the invention relates to an inhibitor of an interaction between phosphatidylserine and a TIM receptor for use for preventing or treating a viral infection, in particular a phosphatidylserine (PtdSer) harboring virus infection such as a flavivirus infection, wherein said inhibitor is preferably (i) a TIM receptor inhibitor, and/or (ii) a phosphatidylserine binding protein. Preferably, said interaction is a direct interaction.

By “a phosphatidylserine harboring virus infection” is meant in particular a “flavivirus infection”. By “flavivirus infection” it is meant an infection with a Dengue virus (DV), a West Nile virus, a tick-borne encephalitis virus, a Saint-Louis encephalitis virus, a Japanese encephalitis virus or a yellow fever virus. Preferably, said TIM receptor is TIM-1, TIM-3 or TIM-4. Preferably, said TIM receptor inhibitor is an anti-TIM receptor antibody, an antisense nucleic acid, a mimetic or a variant TIM receptor, and preferably said TIM receptor inhibitor is a siRNA. Preferably, said phosphatidylserine binding protein is an anti-phosphatidylserine antibody or Annexin 5.

Also provided is a pharmaceutical composition comprising an inhibitor of an interaction between phosphatidylserine and a TIM receptor and additionally at least one other antiviral compound. Preferably, said at least one other antiviral compound is an inhibitor of an interaction of phosphatidylserine and a TAM receptor.

Further provided is the use of an inhibitor of an interaction between phosphatidylserine and a TIM receptor in a method of inhibiting entry of a virus, in particular a PtdSer harboring virus such as a flavivirus, into a cell.

Also provided is a method for preventing or treating a viral infection, in particular a PtdSer harboring virus infection such as a flavivirus infection, comprising administering to an individual in need thereof a therapeutically effective amount of an inhibitor of an interaction between phosphatidylserine and a TIM receptor.

Also provided is the use of an inhibitor of an interaction between phosphatidylserine and a TIM receptor for the manufacture of a medicament for preventing or treating a viral infection, in particular a PtdSer harboring virus infection, in particular a flavivirus infection.

### DEFINITION

By “a phosphatidylserine harboring virus infection” is meant an infection with an enveloped virus that expresses or incorporates PtdSer in its membrane. Prior to infection, the PtdSer is exposed on the viral membrane to receptors of the host cell. Examples of enveloped viruses harboring PtdSer include, but are not limited to: Flavivirus (such as Dengue Virus, West Nile Virus, Yellow Fever Virus), Alphavirus (e.g. Chikungunya Virus), Filovirus (e.g. Ebola Virus), Poxivirus (e.g. Cowpox Virus) and Arenavirus (e.g. Lassa Virus).

“A phosphatidylserine harboring virus infection” may include, for example, a “flavivirus infection”. By “flavivirus infection” it is meant an infection with a Dengue virus (DV), a West Nile virus, a tick-borne encephalitis virus, a Saint-Louis encephalitis virus, a Japanese encephalitis virus or a yellow fever virus (Sabin et al., 1952, A.B. Am. J. Trop. Med. Hyg. 1:30-50; Hammon et al., 1960, Trans. Assoc. Am. Physicians 73:140-155; Smithburn, 1940, Am. J. Trop. Med., 20:471-492; Monath and Heinz, 1996, Flaviviruses, Fields Virology, 3^{rd} edition, p. 961-1034; Gould and Solomon, 2008, Lancet, 371:500-509). The Dengue virus may be of any serotype, i.e. serotype 1, 2, 3 or 4.

By “interaction between phosphatidylserine and a TIM receptor” is meant the direct interaction between phosphatidylserine present at the surface of the PtdSer harboring virus and a TIM receptor present at the surface of the host cell. In fact, the inventors have found that the direct interaction between phosphatidylserine and TIM receptor permits the PtdSer-harboring virus infection or entry into the host cells.

By “inhibitor” is meant an agent that is able to reduce or to abolish the interaction between phosphatidylserine and a TIM receptor. Said inhibitor may also be able to reduce or abolish the expression of a TIM receptor. According to the invention, said inhibitor is (i) a TIM receptor inhibitor and/or (iii) a phosphatidylserine binding protein.

Preferably, said inhibitor is able to reduce or to abolish the interaction between phosphatidylserine and a TIM receptor, by at least 10, 20, 30, 40%, more preferably by at least 50, 60, 70%, and most preferably by at least 75, 80, 85, 90, 95, 96, 97, 98, 99, or 100%.

Reference herein to polypeptides and nucleic acid includes both the amino acid sequences and nucleic acid sequences disclosed herein and variants of said sequences.

Variant proteins may be naturally occurring variants, such as splice variants, alleles and isoforms, or they may be produced by recombinant means. Variations in amino acid sequence may be introduced by substitution, deletion or insertion of one or more codons into the nucleic acid sequence encoding the protein that results in a change in the amino acid sequence of the protein. Optionally the variation is by substitution of 1, 2, 3, 4, 5, 6, 7, 8, 9, 10, 11, 12, 13, 14, 15, 16, 17, 18, 19, 20 or more amino acids with any other amino acid in the protein. Additionally or alternatively, the variation may be by addition or deletion of 1, 2, 3, 4, 5, 6, 7, 8, 9, 10, 11, 12, 13, 14, 15, 16, 17, 18, 19, 20 or more amino acids within the protein.

Variant nucleic acid sequences include sequences capable of specifically hybridizing to the sequence of SEQ ID Nos: 1-4, 6-8, 11, 14, 15, 17, 19, 22, 23, 25, 29-31, 32-35 under moderate or high stringency conditions. Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C.

Fragments of the proteins and variant proteins disclosed herein are also encompassed by the invention. Such fragments may be truncated at the N-terminus or C-terminus, or may lack internal residues, for example, when compared with a full length protein. Certain fragments lack amino acid residues that are not essential for enzymatic activity. Preferably, said fragments are at least about 10, 20, 30, 40, 50, 60, 70, 80, 90, 100, 110, 120, 150, 250, 300, 350, 400, 450, 500 or more amino acids in length.

Fragments of the nucleic acid sequences and variants disclosed herein are also encompassed by the invention. Such fragments may be truncated at 3′ or 5′ end, or may lack internal bases, for example, when compared with a full length nucleic acid sequence. Preferably, said fragments are at least about 10, 20, 30, 40, 50, 60, 70, 80, 90, 100, 110, 120, 150, 250, 300, 350, 400, 450, 500 or more bases in length.

Variant proteins may include proteins that have at least about 80% amino acid sequence identity with a polypeptide sequence disclosed herein. Preferably, a variant protein will have at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length polypeptide sequence or a fragment of a polypeptide sequence as disclosed herein. Amino acid sequence identity is defined as the percentage of amino acid residues in the variant sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both.

Variant nucleic acid sequences may include nucleic acid sequences that have at least about 80% amino acid sequence identity with a nucleic acid sequence disclosed herein. Preferably, a variant nucleic acid sequences will have at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length nucleic acid sequence or a fragment of a nucleic acid sequence as disclosed herein. Nucleic acid acid sequence identity is defined as the percentage of nucleic acids in the variant sequence that are identical with the nucleic acids in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the context of the present application, the percentage of identity is calculated using a global alignment (i.e. the two sequences are compared over their entire length). Methods for comparing the identity of two or more sequences are well known in the art. The <<needle>> program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS:needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95%, 96%, 97%, 98% or 99% identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

Amino acid substitutions may be conservative or non-conservative. Preferably, substitutions are conservative substitutions, in which one amino acid is substituted for another amino acid with similar structural and/or chemical properties. The substitution preferably corresponds to a conservative substitution as indicated in the table below.

| Conservative substitutions | Type of Amino Acid |
|---|---|
| Ala, Val, Leu, Ile, Met, Pro, Phe, | Amino acids with aliphatic |
| Trp | hydrophobic side chains |
| Ser, Tyr, Asn, Gln, Cys | Amino acids with uncharged but |
| | polar side chains |
| Asp, Glu | Amino acids with acidic side chains |
| Lys, Arg, His | Amino acids with basic side chains |
| Gly | Neutral side chain |

The term “antibody” refers to immunoglobulin molecules and immunologically active portions of immunoglobulin molecules, i.e., molecules that contain an antigen binding site which immunospecifically binds an antigen. As such, the term antibody encompasses not only whole antibody molecules, but also antibody fragments as well as variants of antibodies, including derivatives such as humanized antibodies. In natural antibodies, two heavy chains are linked to each other by disulfide bonds and each heavy chain is linked to a light chain by a disulfide bond. There are two types of light chain, lambda (A) and kappa (K). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non hypervariable or framework regions (FR) influence the overall domain structure and hence the combining site. Complementarity determining regions (CDRs) refer to amino acid sequences which, together, define the binding affinity and specificity of the natural Fv region of a native immunoglobulin binding-site. The light and heavy chains of an immunoglobulin each have three CDRs, designated L-CDR1, L-CDR2, L-CDR3 and H-CDR1, H-CDR2, H-CDR3, respectively. Therefore, an antigen-binding site includes six CDRs, comprising the CDR set from each of a heavy and a light chain V region.

Framework Regions (FRs) refer to amino acid sequences interposed between CDRs, i.e. to those portions of immunoglobulin light and heavy chain variable regions that are relatively conserved among different immunoglobulins in a single species, as defined by Kabat, et al (Sequences of Proteins of Immunological Interest (National Institutes of Health, Bethesda, Md., 1991). As used herein, a “human framework region” is a framework region that is substantially identical (about 85%, or more, in particular 90%, 95%, or 100%) to the framework region of a naturally occurring human antibody.

The term “monoclonal antibody” or “mAb” as used herein refers to an antibody molecule of a single amino acid composition, that is directed against a specific antigen and which may be produced by a single clone of B cells or hybridoma. Monoclonal antibodies may also be recombinant, i.e. produced by protein engineering.

The term “chimeric antibody” refers to an engineered antibody which comprises a VH domain and a VL domain of an antibody derived from a non-human animal, in association with a CH domain and a CL domain of another antibody, in particular a human antibody. As the non-human animal, any animal such as mouse, rat, hamster, rabbit or the like can be used. A chimeric antibody may also denote a multispecific antibody having specificity for at least two different antigens.

The term “humanized antibody” refers to antibodies in which the framework or “complementarity determining regions” (CDR) have been modified to comprise the CDR from a donor immunoglobulin of different specificity as compared to that of the parent immunoglobulin. In a preferred embodiment, a mouse CDR is grafted into the framework region of a human antibody to prepare the “humanized antibody”.

“Antibody fragments” comprise a portion of an intact antibody, preferably the antigen binding or variable region of the intact antibody. Examples of antibody fragments include Fv, Fab, F(ab′)2, Fab′, dsFv, scFv, sc(Fv)2, diabodies and multispecific antibodies formed from antibody fragments.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)₂” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab” refers to an antibody fragment having a molecular weight of about 50,000 and antigen binding activity, which is obtained by cutting a disulfide bond of the hinge region of the F(ab′)₂.

A single chain Fv (“scFv”) polypeptide is a covalently linked VH:VL heterodimer which is usually expressed from a gene fusion including VH and VL encoding genes linked by a peptide-encoding linker. The human scFv fragment of the invention includes CDRs that are held in appropriate conformation, preferably by using gene recombination techniques. “dsFv” is a VH:VL heterodimer stabilised by a disulphide bond. Divalent and multivalent antibody fragments can form either spontaneously by association of monovalent scFvs, or can be generated by coupling monovalent scFvs by a peptide linker, such as divalent sc(Fv)₂.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993, Nature 365, 566) interactions and alters the activity of the target RNA (for a review, see Stein and Cheng, 1993, Science 261, 1004, and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to 2, 3, 4, 5, 6, 7, 8, 9, 10 or more non-contiguous substrate sequences or 2, 3, 4, 5, 6, 7, 8, 9, 10 or more non-contiguous sequence portions of an antisense molecule can be complementary to a target sequence or both (for example, see Crooke, 2000, Methods Enzymol., 313, 3-45). In addition, antisense DNA can be used to target RNA by means of DNA-RNA interactions, thereby activating RNAse H, which digests the target RNA in the duplex. The antisense oligonucleotides can comprise one or more RNAse H activating region, which is capable of activating RNAse H cleavage of a target RNA.

Upon introduction, the antisense nucleic acid enters a cellular pathway that is commonly referred to as the RNA interference (RNAi) pathway. The term “RNA interference” or “RNAi” refers to selective intracellular degradation of RNA also referred to as gene silencing. RNAi also includes translational repression by small interfering RNAs (siRNAs). RNAi can be initiated by introduction of Long double-stranded RNA (dsRNAs) or siRNAs or production of siRNAs intracellularly, eg from a plasmid or transgene, to silence the expression of one or more target genes. Alternatively RNAi occurs in cells naturally to remove foreign RNAs, eg viral RNAs. Natural RNAi proceeds via dicer directed fragmentation of precursor dsRNA which direct the degradation mechanism to other cognate RNA sequences.

In some embodiments, the antisense nucleic acid may be Long double-stranded RNAs (dsRNAs), microRNA (miRNA) and/or small interferent RNA (siRNA).

As used herein “Long double-stranded RNA” or “dsRNA” refers to an oligoribonucleotide or polyribonucleotide, modified or unmodified, and fragments or portions thereof, of genomic or synthetic origin or derived from the expression of a vector, which may be partly or fully double stranded and which may be blunt ended or contain a 5′ and or 3′ overhang, and also may be of a hairpin form comprising a single oligoribonucleotide which folds back upon itself to give a double stranded region. In some embodiments, the dsRNA has a size ranging from 150 bp to 3000 bp, preferably ranging from 250 bp to 2000 bp, still more preferably ranging from 300 bp to 1000 bp. In some embodiments, said dsRNA has a size of at least 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950, 1000, 1500 bp. In some embodiments, said dsRNA has a size of at most 3000, 2500, 2000, 1500, 1000, 950, 900, 850, 800, 750, 700, 650, 600, 550, 500, 450, 400, 350, 300 bp.

A “small interfering RNA” or “siRNA” is a RNA duplex of nucleotides that is targeted to a gene interest. A RNA duplex refers to the structure formed by the complementary pairing between two regions of a RNA molecule. siRNA is targeted to a gene in that the nucleotide sequence of the duplex portion of the siRNA is complementary to a nucleotide sequence of the targeted gene. In some embodiments, the length of the duplex of siRNAs is ranging from 15 nucleotides to 50 nucleotides, preferably ranging from 20 nucleotides to 35 nucleotides, still more preferably ranging from 21 nucleotides to 29 nucleotides. In some embodiments, the duplex can be of at least 15, 16, 17, 18, 19, 20, 21, 22, 23, 24, 25, 26, 27, 28, 29, 30, 31, 32, 33, 34, 35, 40, 45, 50 nucleotides in length. In some embodiments, the duplex can be of at most 45, 40, 35, 34, 33, 32, 31, 30, 29, 28, 27, 26, 25, 24, 23, 22, 21, 20, 19, 18, 17, 16, 15 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, or 13 nucleotides in length. The hairpin structure can also contain 3 or 5 overhang portions. In some embodiments, the overhang is a 3′ or a 5′ overhang 0, 1, 2, 3, 4, or 5 nucleotides in length.

Injection and transfection antisense nucleic acid into cells and organisms has been the main method of delivery. However, expression vectors may also be used to continually express antisense nucleic acid in transiently and stably transfected mammalian cells. (See for example, e.g., Brummelkamp et al., 2002, Science, 296:550-553; Paddison et al., 2002, Genes & Dev, 16:948-958).

Antisense nucleic acid may be synthesized chemically or expressed via the use of a single stranded DNA expression vector or equivalent thereof using protocols known in the art as described for example in Caruthers et al., 1992, Methods in Enzymology, 211:3-19; International PCT Publication No. WO 99/54459; Brennan et al., 1998, Biotechnol Bioeng, 61:33-45; and U.S. Pat. No. 6,001,311. In a non-limiting example, small scale syntheses are conducted on a 394 Applied Biosystems, Inc. synthesizer. Alternatively, the antisense nucleic acid molecules of the present invention can be synthesized separately and joined together post-synthetically, for example by ligation (International PCT publication No. WO 93/23569; Bellon et al., 1997, Bioconjugate Chem, 8:204).

The antisense nucleic acid of the invention may be able of decreasing the expression of the targeted gene, for example a TIM receptor, by at least 10, 20, 30, 40%, more preferably by at least 50, 60, 70%, and most preferably by at least 75, 80, 85, 90, 95, 96, 97, 98, 99, 100%.

By “variant TIM receptor” or “variant TAM receptor” or “variant Gas6 protein” is respectively meant a receptor that differs from the TIM receptor or the TAM receptor or the Gas6 protein by one or several amino acid(s). For example, said variant TIM receptor may differ from the TIM receptor in that it is no longer able to bind to the phosphatidylserine or in that it is no longer able to have its kinase activity. For example, said variant TAM receptor may differ from the TAM receptor in that it is no longer able to bind to the Gas6 protein, such as for example an AXL receptor of sequence SEQ ID NO: 20 or 21 carrying the mutation E63R, E66R or T847R, or in that it is no longer able to have its kinase activity, such as for example an AXL receptor of sequence SEQ ID NO: 20 carrying the mutation K558M, or an AXL receptor of sequence SEQ ID NO: 21 carrying the mutation K567M. For example, said variant Gas6 protein may differ from the Gas6 protein in that it is no longer able to bind to phosphatidylserine and/or to a TAM receptor. For example, said variant Gas6 protein may be the Gas6Δgla (also named rmGas6Δgla) of sequence SEQ ID NO: 36.

The terms “subject”, “individual” or “host” are used interchangeably and may be, for example, a human or a non-human mammal. For example, the subject is a bat; a ferret; a rabbit; a feline (cat); a canine (dog); a primate (monkey), an equine (horse); a human, including man, woman and child.

### Inhibitor of Interaction Between Phosphatidylserine and a TIM Receptor

Phosphatidylserine is a phospholipid which phosphate group is associated to the serine amino acid and which is referenced under the CAS number 8002-43-5.

By “TIM receptor” is meant a tyrosine kinase receptor of the T-cell Immunoglobulin Mucin (TIM) family. In preferred embodiments, said TIM receptor is a TIM-1, TIM-3 or TIM-4.

In some embodiments, the TIM-1 receptor comprises or consists of:
a) the sequence SEQ ID NO: 5 (GenBank Number AAH13325.1, update Oct. 4, 2003),
b) the sequence encoded by the nucleic acid SEQ ID NO: 6 (NCBI Reference Sequence NM_{—}012206.2, update Nov. 26, 2011),
c) the sequence encoded by the nucleic acid SEQ ID NO: 7 (NCBI Reference Sequence NM_{—}001099414.1, update Nov. 26, 2011),
d) the sequence encoded by the nucleic acid SEQ ID NO: 8 (NCBI Reference Sequence NM_{—}001173393.1, update Dec. 4, 2011),
e) a sequence at least 80, 85, 90, 95, 96, 97, 98, 99% identical to the sequence of a) to d).

In some embodiments, the TIM-3 receptor comprises or consists of:
a) the sequence SEQ ID NO: 9 (Gen Bank Number AAH20843.1, update Sep. 16, 2003),
b) the sequence SEQ ID NO: 10 (GenBank Number AAH63431.1, update Jul. 15, 2006),
c) the sequence encoded by the nucleic acid SEQ ID NO: 11 (NCBI Reference Sequence NM_{—}032782.4, update Dec. 25, 2011),
d) a sequence at least 80, 85, 90, 95, 96, 97, 98, 99% identical to the sequence of a) to c).

In some embodiments, the TIM-4 receptor comprises or consists of:
a) the sequence SEQ ID NO: 12 (NCBI Reference Sequence NP_{—}612388.2, update Dec. 24, 2011),
b) the sequence SEQ ID NO: 13 (NCBI Reference Sequence NP_{—}001140198.1, update Dec. 25, 2011),
c) the sequence encoded by the nucleic acid SEQ ID NO: 14 (NCBI Reference Sequence NM_{—}138379.2, update Dec. 24, 2011),
d) the sequence encoded by the nucleic acid SEQ ID NO: 15 (NCBI Reference Sequence NM_{—}001146726.1, update Dec. 25, 2011),
e) a sequence at least 80, 85, 90, 95, 96, 97, 98, 99%, identical to the sequence of a) to d).

In some embodiments, the TIM receptor inhibitor is an anti-TIM receptor antibody, an antisense nucleic acid, a mimetic or a variant TIM receptor.

Preferably, said TIM receptor inhibitor is an antisense nucleic acid, and more preferably said TIM receptor inhibitor is a siRNA. Said antisense nucleic acid may comprise or consist of a sequence that is able to inhibit or reduce the expression of a TIM receptor of sequence SEQ ID NO: 5, 9, 10, 12, or 13, or a TIM receptor of sequence encoded by the nucleic acid SEQ ID NO: 6, 7, 8, 11, 14 or 15. Said antisense nucleic acid may comprise or consist of a sequence complementary to a nucleic acid encoding a TIM receptor, for example a nucleic acid of sequence SEQ NO: 6, 7, 8, 11, 14 or 15. In one embodiment, said siRNA comprises or consists of at least one siRNA of sequence SEQ ID NO: 1, 2, 3, or 4. In one embodiment, said siRNA comprises or consists of at least 2, 3, or 4 siRNA selected from the group consisting of SEQ ID NOs: 1, 2, 3, and 4. In one embodiment, said siRNA comprises or consists of at most 4, 3, or 2 siRNA selected from the group consisting of SEQ ID NOs: 1, 2, 3, and 4. In one embodiment, said siRNA comprises or consists of the four siRNA of sequence SEQ ID NO: 1, 2, 3, and 4.

Preferably, said anti-TIM receptor antibody is the anti-TIM1 receptor antibody ARD5 described in Kondratowicz et al., 2011, PNAS, 108:8426-8431, or the anti-TIM1 antibody A6G2 described in Sonar et al., 2010, The Journal of Clinical investigation, 120: 2767-2781.

Preferably, said mimetic comprises or consists of the extracellular domain of the TIM receptor. For example, said mimetic may comprise or consist of the amino acid sequence of residues 21 to 295 for TIM-1 of SEQ ID NO: 5, said mimetic may comprise or consist of the amino acid sequence of residues 21 to 290 for TIM-1 of SEQ ID NO: 47 or said mimetic may comprise or consist of the amino acid sequence of residues 25 to 314 for TIM-4 of SEQ ID NO: 12.

Preferably, said anti-TIM receptor antibody is an antibody directed against the binding site of the TIM receptor to phosphatidylserine. Preferably, said antibody directed against the binding site of the TIM receptor to phosphatidylserine is directed to the Metal Ion-dependent Ligand Binding Site (MILIB) of the TIM receptor. Still more preferably, said anti-TIM receptor is directed to the amino acids 111 to 115 of sequence SEQ ID NO: 5, or to the amino acids 119 to 122 of sequence SEQ ID NO: 12 or SEQ ID NO: 13.

In some embodiments, the phosphatidylserine binding protein may be an anti-phosphatidylserine antibody or a protein that is able to bind to the phosphatidylserine, thereby blocking the interaction between phosphatidylserine and a TIM receptor. For example, said antibody may be the anti-phosphatidylserine antibody clone 1H6 (Upstate®).

Preferably, said anti-phosphatidylserine antibody is an antibody directed against the binding site of phosphatidylserine to the TIM receptor.

Preferably, said phosphatidylserine binding protein is the Annexin V. Preferably, said Annexin V protein comprises or consists of:
a) the sequence SEQ ID NO: 16 (NCBI Reference Sequence NP_{—}001145.1, update Feb. 1, 2012),
b) the sequence encoded by the nucleic acid of sequence SEQ ID NO: 17 (NCBI

Reference Sequence NM_{—}001154.3, update Dec. 18, 2011),
c) a sequence at least 80, 85, 90, 95, 96, 97, 98, 99% identical to the sequence of a) or b).

### Antiviral Compounds

In a preferred embodiment, the inhibitor according to the invention is for administration in combination with at least one other antiviral compound, either sequentially or simultaneously.

Sequential administration indicates that the components are administered at different times or time points, which may nonetheless be overlapping. Simultaneous administration indicates that the components are administered at the same time.

The antiviral compound may include, but is not limited to, neuraminidase inhibitors, viral fusion inhibitors, protease inhibitors, DNA polymerase inhibitors, signal transduction inhibitors, reverse transcriptase inhibitors, interferons, nucleoside analogs, integrase inhibitors, thymidine kinase inhibitors, viral sugar or glycoprotein synthesis inhibitors, viral structural protein synthesis inhibitors, viral attachment and adsorption inhibitors, viral entry inhibitors and their functional analogs.

Neuraminidase inhibitors may include oseltamivir, zanamivir and peramivir. Viral fusion inhibitors may include cyclosporine, maraviroc, enfuviritide and docosanol.

Protease inhibitors may include saquinavir, indinarvir, amprenavir, nelfinavir, ritonavir, tipranavir, atazanavir, darunavir, zanamivir and oseltamivir.

DNA polymerase inhibitors may include idoxuridine, vidarabine, phosphonoacetic acid, trifluridine, acyclovir, forscarnet, ganciclovir, penciclovir, cidoclovir, famciclovir, valaciclovir and valganciclovir.

Signal transduction inhibitors include resveratrol and ribavirin. Nucleoside reverse transcriptase inhibitors (NRTIs) may include zidovudine (ZDV, AZT), lamivudine (3TC), stavudine (d4T), zalcitabine (ddC), didanosine (2′,3′-dideoxyinosine, ddl), abacavir (ABC), emirivine (FTC), tenofovir (TDF), delaviradine (DLV), fuzeon (T-20), indinavir (IDV), lopinavir (LPV), atazanavir, combivir (ZDV/3TC), kaletra (RTV/LPV), adefovir dipivoxil and trizivir (ZDV/3TC/ABC). Non-nucleoside reverse transcriptase inhibitors (NNRTIs) may include nevirapine, delavirdine, UC-781 (thiocarboxanilide), pyridinones, TIBO, calanolide A, capravirine and efavirenz.

Viral entry inhibitors may include Fuzeon (T-20), NB-2, NB-64, T-649, T-1249, SCH-C, SCH-D, PRO 140, TAK 779, TAK-220, RANTES analogs, AK602, UK-427, 857, monoclonal antibodies against relevant receptors, cyanovirin-N, clyclodextrins, carregeenans, sulfated or sulfonated polymers, mandelic acid condensation polymers, AMD-3100, and functional analogs thereof.

Preferably, said at least one other antiviral compound is an inhibitor of an interaction between phosphatidylserine and a TAM receptor.

In some embodiments, said inhibitor of interaction of phosphatidylserine and a TAM receptor is a TAM receptor inhibitor and/or a Gas6 inhibitor.

By “TAM receptor”, it is meant a TYRO-3, AXL or MER receptor.

Preferably, the TYRO-3 receptor comprises or consists of:
a) the sequence SEQ ID NO: 18 (NCBI Reference Sequence NP_{—}006284.2, update Nov. 14, 2011),
b) the sequence encoded by the nucleic acid of sequence SEQ ID NO: 19 (NCBI Reference Sequence NM_{—}006293.3, update Jan. 14, 2012),
c) a sequence at least 80, 85, 90, 95, 96, 97, 98, 99% identical to the sequence of a) or b).

Preferably, the AXL receptor comprises or consists of:
a) the sequence SEQ ID NO: 20 (NCBI Reference Sequence NP_{—}001690.2, update Nov. 26, 2011),
b) the sequence SEQ ID NO: 21 (NCBI Reference Sequence NP_{—}068713.2, update Nov. 26, 2011),
c) the sequence encoded by the nucleic acid of sequence SEQ ID NO: 22 (NCBI Reference Sequence NM_{—}021913.3, update Jan. 15, 2012),
d) the sequence encoded by the nucleic acid of sequence SEQ ID NO: 23 (NCBI Reference Sequence NM_{—}001699.4, update Jan. 15, 2012),
e) a sequence at least 80, 85, 90, 95, 96, 97, 98, 99% identical to the sequence of a) to d).

Preferably, the MER receptor comprises or consists of:
a) the sequence SEQ ID NO: 24 (NCBI Reference Sequence NP_{—}006334.2, update Dec. 24, 2011),
b) the sequence encoded by the nucleic acid of sequence SEQ ID NO: 25 (NCBI Reference Sequence NM_{—}006343.2, update Dec. 24, 2011),
c) a sequence at least 80, 85, 90, 95, 96, 97, 98, 99% identical to the sequence of a) or b).

The Gas6 protein is a bridge molecule that mediates the interaction between phosphatidylserine and a TAM receptor.

Preferably, the Gas6 protein comprises or consists of:
a) the sequence SEQ ID NO: 26 (NCBI Reference Sequence NP_{—}000811.1, update Dec. 24, 2011),
b) the sequence SEQ ID NO: 27 (NCBI Reference Sequence NP_{—}001137417.1, update Dec. 24, 2011),
c) the sequence SEQ ID NO: 28 (NCBI Reference Sequence NP_{—}001137418.1, update Dec. 24, 2011),
d) the sequence encoded by the nucleic acid of sequence SEQ ID NO: 29 (NCBI Reference Sequence NM_{—}000820.2, update Jan. 15, 2012),
e) the sequence encoded by the nucleic acid of sequence SEQ ID NO: 30 (NCBI Reference Sequence NM_{—}001143945.1, update Jan. 15, 2012),
f) the sequence encoded by the nucleic acid of sequence SEQ ID NO: 31 (NCBI Reference Sequence NM_{—}001143946.1, update Jan. 15, 2012),
g) a sequence at least 80, 85, 90, 95, 96, 97, 98, 99% identical to the sequence of a) to f).

In some embodiments, the TAM receptor inhibitor is an anti-TAM receptor antibody, an antisense nucleic acid, a mimetic or a variant TAM receptor.

Preferably, said TAM receptor inhibitor is an antisense nucleic acid, and more preferably said TAM receptor inhibitor is a siRNA. Said antisense nucleic acid may comprise or consist of a sequence that is able to inhibit or reduce the expression of a TAM receptor of sequence SEQ ID NO: 18, 20, 21, or 24, or a TAM receptor of sequence encoded by the nucleic acid SEQ ID NO: 19, 22, 23, or 25. Said antisense nucleic acid may comprise or consist of a sequence complementary to a nucleic acid encoding a TAM receptor, for example a nucleic acid of sequence SEQ ID NO: 19, 22, 23, or 25. In one embodiment, said siRNA comprises or consists of at least One siRNA of sequence SEQ ID NO: 32, 33, 34 or 35. In one embodiment, said siRNA comprises or consists of at least 2, 3, or 4 siRNA selected from the group consisting of SEQ ID NOs: 32, 33, 34, and 35. In one embodiment, said siRNA comprises or consists of at most 4, 3, 2, or 1 siRNA selected from the group consisting of SEQ ID NOs: 32, 33, 34, and 35. In one embodiment, said siRNA comprises or consists of the four siRNA of sequence SEQ ID NO: 32, 33, 34, and 35.

Preferably, said mimetic comprises or consists of the extracellular domain of the TAM receptor. For example, said mimetic may comprise or consist of the amino acids 26 to 451 of SEQ ID NO: 20 or SEQ ID NO: 21.

Still more preferably, said mimetic comprises or consists of the soluble form of the extracellular domain of the TAM receptor. For example, said mimetic may comprise or consist of the sequence of amino acids 41 to 428 of SEQ ID NO: 18, or of the sequence of amino acids 33 to 440 of SEQ ID NO: 20 or SEQ ID NO: 21.

Preferably, said anti-TAM receptor antibody is an antibody directed against the binding site of the TAM receptor to the Gas6 protein. Preferably, said anti-TAM receptor antibody is directed to the amino acids 63 to 84 of the sequence SEQ ID NO: 20 or SEQ ID NO: 21.

In some embodiments, the Gas6 inhibitor is an anti-Gas6 antibody, an antisense nucleic acid, a mimetic or a variant Gas6 protein.

Preferably, said Gas6 inhibitor is an antisense nucleic acid, and more preferably said Gas6 inhibitor is a siRNA. Said antisense nucleic acid may comprise or consist of a sequence that is able to inhibit or reduce the expression of a Gas6 protein of sequence SEQ ID NO: 26, 27, or 28, or a Gas6 protein of sequence encoded by the nucleic acid SEQ ID NO: 29, 30, or 31. Said antisense nucleic acid may comprise or consist of a sequence complementary to a nucleic acid encoding Gas6 or fragment thereof, for example a nucleic acid of sequence SEQ NO: 29, 30, or 31.

Preferably, said Gas6 inhibitor is the variant Gas6 protein Gas6ΔGla of sequence SEQ ID NO: 36.

Preferably, said Gas-6 mimetic comprises or consists of the phosphatidylserine recognition site which may comprise or consist of the amino acid sequence of residues 53 to 94 of SEQ ID NO: 26 or said mimetic comprises or consists of the receptor binding site which may comprise or consist of the amino acid sequence of residues 298 to 670 of SEQ ID NO: 26.

Preferably, said anti-Gas6 antibody is an antibody directed against the binding site of the Gas6 protein to the TAM receptor. Preferably, said anti-Gas6 antibody is directed to the amino acids 304 to 312 of the sequence SEQ ID NO: 26, to the amino acids 31 to 39 of the sequence SEQ ID NO: 27, or to the amino acids 5 to 13 of the sequence SEQ ID NO: 28.

### Method for Inhibiting Entry of a Phosphatidylserine Harboring Virus into a Cell

The inhibitor according to the invention may be used in a method of inhibiting entry of a PtdSer harboring virus into a cell.

Said method may be an in vitro or ex vivo method, or a method of prevention or treatment of a PtdSer harboring virus infection as described herein.

The invention thus provides the use of an inhibitor as defined herein in an in vitro or in vivo method for inhibiting entry of a PtdSer harboring virus into a cell. Also provided is an inhibitor as defined herein for use in an in vitro or in vivo method for inhibiting entry of a PtdSer harboring virus into a cell.

In some embodiments, said inhibitor is use in combination with at least one other antiviral compound as defined hereabove.

Said method may comprise, for example, exposing said cell and/or said PtdSer harboring virus to said inhibitor. Where the method is an in vivo method, the method may comprise administering said inhibitor to a subject, preferably a patient in need thereof.

In some embodiments, said cell may be dendritic cells, endothelial cells, astrocytes, hepatocytes, neurons, Kupffer cells, and/or macrophages

### Pharmaceutical Compositions

The inhibitor according to the invention may be formulated in a pharmaceutically acceptable composition, either alone or in combination with the at least one other antiviral compound.

The invention thus provides a pharmaceutical composition comprising an inhibitor according to the invention and additionally at least one other antiviral compound.

Said at least one other antiviral compound may be a compound as defined above.

In one embodiment, said inhibitor comprises or consists of at least 1, 2, 3, or 4, or at most 4, 3, 2, or 1 siRNA selected from the group consisting of siRNA of sequence SEQ ID NOs: 1, 2, 3, and 4, and/or annexin V as defined hereabove, and the at least one other antiviral compound comprises or consists of at least 1, 2, 3, or 4, or at most 4, 3, 2, or 1 siRNA selected from the group consisting of siRNA of sequence SEQ ID NOs: 32, 33, 34, and 35 and/or the variant Gas6 protein Gas6Δgla of sequence SEQ ID NO: 36 as defined hereabove. In one embodiment, said inhibitor comprises or consists of 4 siRNA of sequence SEQ ID NOs: 1, 2, 3, and 4, and/or annexin V as defined hereabove, and the at least one other antiviral compound comprises or consists of 4 siRNA of sequence SEQ ID NOs: 32, 33, 34, and 35 and/or the variant Gas6 protein Gas6Δgla of sequence SEQ ID NO: 36 as defined hereabove.

The pharmaceutical compositions according to the invention may be administered orally in the form of a suitable pharmaceutical unit dosage form. The pharmaceutical compositions of the invention may be prepared in many forms that include tablets, hard or soft gelatin capsules, aqueous solutions, suspensions, and liposomes and other slow-release formulations, such as shaped polymeric gels.

The mode of administration and dosage forms are closely related to the properties of the therapeutic agents or compositions which are desirable and efficacious for the given treatment application. Suitable dosage forms include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and other dosage forms for systemic delivery of active ingredients.

Pharmaceutical compositions of the invention may be administered by any method known in the art, including, without limitation, transdermal (passive via patch, gel, cream, ointment or iontophoretic); intravenous (bolus, infusion); subcutaneous (infusion, depot); transmucosal (buccal and sublingual, e.g., orodispersible tablets, wafers, film, and effervescent formulations; conjunctival (eyedrops); rectal (suppository, enema)); or intradermal (bolus, infusion, depot).

Oral liquid pharmaceutical compositions may be in the form of, for example, aqueous or oily suspensions, solutions, emulsions, syrups or elixirs, or may be presented as a dry product for constitution with water, or other suitable vehicle before use. Such liquid pharmaceutical compositions may contain conventional additives such as suspending agents, emulsifying agents, non-aqueous vehicles (which may include edible oils), or preservatives.

Pharmaceutical compositions of the invention may also be formulated for parenteral administration (e.g., by injection, for example, bolus injection or continuous infusion) and may be presented in unit dosage form in ampoules, pre-filled syringes, small volume infusion containers or multi-dose containers with an added preservative. The pharmaceutical compositions may take such forms as suspensions, solutions, or emulsions in oily or aqueous vehicles, and may contain formulating agents such as suspending, stabilizing and/or dispersing agents. Alternatively, the pharmaceutical compositions of the invention may be in powder form, obtained by aseptic isolation of sterile solid or by lyophilization from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the pharmaceutical composition with the softened or melted carrier(s) followed by chilling and shaping in molds.

For administration by inhalation, the pharmaceutical compositions according to the invention are conveniently delivered from an insufflator, nebulizer or a pressurized pack or other convenient means of delivering an aerosol spray. Pressurized packs may comprise suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the pharmaceutical compositions of the invention may take the form of a dry powder composition, for example, a powder mix of the pharmaceutical composition and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the pharmaceutical compositions of the invention may be administered via a liquid spray, such as via a plastic bottle atomizer. Typical of these are the Mistometerg (isoproterenol inhaler-Wintrop) and the Medihaler® (isoproterenol inhaler-Riker).

For antisense nucleic acid administration, the pharmaceutical compositions of the invention may be prepared in forms that include encapsulation in liposomes, microparticles, microcapsules, lipid-based carrier systems. Non limiting examples of alternative lipid based carrier systems suitable for use in the present invention include polycationic polymer nucleic acid complexes (see, e.g. US Patent Publication No 20050222064), cyclodextrin polymer nucleic acid complexes (see, e.g. US Patent Publication No 20040087024), biodegradable poly 3 amino ester polymer nucleic acid complexes (see, e.g. US Patent Publication No 20040071654), pH sensitive liposomes (see, e.g. US Patent Publication No 20020192274), anionic liposomes (see, e.g. US Patent Publication No 20030026831), cationic liposomes (see, e.g. US Patent Publication No 20030229040), reversibly masked lipoplexes (see, e.g. US Patent Publication No 20030180950), cell type specific liposomes (see, e.g. US Patent Publication No 20030198664), microparticles containing polymeric matrices (see, e.g. US Patent Publication No 20040142475), pH sensitive lipoplexes (see, e.g. US Patent Publication No 20020192275), liposomes containing lipids derivatized with releasable hydrophilic polymers (see, e.g. US Patent Publication No 20030031704), lipid en trapped nucleic acid (see, e.g. PCT Patent Publication No WO 03/057190), lipid encapsulated nucleic acid (see, e.g. US Patent Publication No 20030129221), polycationic sterol derivative nucleic acid complexes (see, e.g. U.S. Pat. No. 6,756,054), other liposomal compositions (see, e.g. US Patent Publication No 20030035829), other microparticle compositions (see, e.g. US Patent Publication No 20030157030), poly-plexes (see, e.g. PCT Patent Publication No WO 03/066069), emulsion compositions (see, e.g. U.S. Pat. No. 6,747,014), condensed nucleic acid complexes (see, e.g. US Patent Publication No 20050123600), other polycationic nucleic acid complexes (see, e.g. US Patent Publication No 20030125281), polyvinylether nucleic acid complexes (see, e.g. US Patent Publication No 20040156909), polycyclic amidinium nucleic acid complexes (see, e.g. US Patent Publication No 20030220289), nanocapsule and microcapsule compositions (see, e.g. PCT Patent Publication No WO 02/096551), stabilized mixtures of liposomes and emulsions (see, e.g. EP1304160), porphyrin nucleic acid complexes (see, e.g. U.S. Pat. No. 6,620,805), lipid nucleic acid complexes (see, e.g. US Patent Publication No 20030203865), nucleic acid micro emulsions (see, e.g. US Patent Publication No 20050037086), and cationic lipid based compositions (see, e.g. US Patent Publication No 20050234232). One skilled in the art will appreciate that modified siRNA of the present invention can also be delivered as a naked siRNA molecule.

Pharmaceutical compositions of the invention may also contain other adjuvants such as flavorings, colorings, anti-microbial agents, or preservatives.

It will be further appreciated that the amount of the pharmaceutical compositions required for use in treatment will vary not only with the therapeutic agent selected but also with the route of administration, the nature of the condition being treated and the age and condition of the patient and will be ultimately at the discretion of the attendant physician or clinician.

### Administration and Methods of Treatment

The invention also relates to a method for preventing or treating a PtdSer harboring virus infection in an individual in need thereof comprising administering a therapeutically effective amount of an inhibitor according to the invention.

By “treatment” is meant a therapeutic use (i.e. on a patient having a given disease) and by “preventing” is meant a prophylactic use (i.e. on an individual susceptible of developing a given disease). The term “treatment” not only includes treatment leading to complete cure of the disease, but also treatments slowing down the progression of the disease and/or prolonging the survival of the patient.

An “effective amount” refers to an amount effective, at dosages and for periods of time necessary, to achieve the desired therapeutic or prophylactic result.

A therapeutically effective amount of an inhibitor of the invention may vary according to factors such as the disease state, age, sex, and weight of the individual, and the ability of the protein, to elicit a desired therapeutic result. A therapeutically effective amount encompasses an amount in which any toxic or detrimental effects of the inhibitor are outweighed by the therapeutically beneficial effects. A therapeutically effective amount also encompasses an amount sufficient to confer benefit, e.g., clinical benefit.

In the context of the present invention, “preventing a phosphatidylserine harboring virus infection” may mean prevention of a PtdSer harboring virus infection or entry into the host cell.

In the context of the present invention, “treating a phosphatidylserine harboring virus infection”, may mean reversing, alleviating, or inhibiting phosphatidylserine harboring virus infection or entry into the host cell.

In the context of the invention, phosphatidylserine harboring virus infection may be reduced by at least 30, 35, 40, 45, 50, 55, 60, 65, 70, 80, 85, 90, 91, 92, 93, 94, 95, 96, 97, 98, 99, 100%.

In some embodiments, the methods of the invention comprise the administration of an inhibitor as defined above, in combination with at least one other antiviral compound as defined above, either sequentially or simultaneously. For example, said at least one other antiviral compound is an inhibitor of an interaction between phosphatidylserine and a TAM receptor as defined hereabove.

In another embodiment, said method comprises the administration of a pharmaceutical composition according to the invention.

The administration regimen may be a systemic regimen. The mode of administration and dosage forms are closely related to the properties of the therapeutic agents or compositions which are desirable and efficacious for the given treatment application. Suitable dosage forms and routes of administration include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and/or other dosage forms and routes of administration for systemic delivery of active ingredients. In a preferred embodiment, the dosage forms are for parenteral administration.

The administration regimen may be for instance for a period of at least 5, 6, 7, 8, 9, 10, 15, 20, 25, 30, 35, 40, 45, 50, 55, 60, 65, 70, 75, 80, 85, 90, 95, 100 days.

The dose range may be between 0.1 mg/kg/day and 100 mg/kg/day. More preferably, the dose range is between 0.5 mg/kg/day and 100 mg/kg/day. Most preferably, the dose range is between 1 mg/kg/day and 80 mg/kg/day. Most preferably, the dose range is between 5 mg/kg/day and 50 mg/kg/day, or between 10 mg/kg/day and 40 mg/kg/day.

In some embodiments, the dose may be of at least 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50 mg/kg/day. In some embodiments, the dose may be of at most 50, 45, 40, 35, 30, 25, 20, 25, 15, 10, 5, 1, 0.5, 0.1 mg/kg/day.

The dose range may also be between 10 to 10000 UI/kg/day. More preferably, the dose range is between 50 to 5000 UI/kg/day, or between 100 to 1000 UI/kg/day.

In some embodiments, the dose may be of at least 10, 25, 50, 75, 100, 150, 200, 250, 300, 350, 400, 450, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000 UI/kg/day. In some embodiments, the dose may be of at most 10000, 9500, 9000, 8500, 8000, 7500, 7000, 6500, 6000, 5500, 5000, 4500, 4000, 3500, 3000, 2500, 2000, 1500, 1000, 900, 800, 600, 500, 450, 400, 350, 300, 250, 200, 150, 100 UI/kg/day.

The invention will now be described in more detail with reference to the following figures and examples. All literature and patent documents cited herein are hereby incorporated by reference.

### SEQUENCE LISTING

SEQ ID NO: 1 shows the sequence of the siRNA 5′-AAACUCAACUGUUCCUACA-3′ against TIM-1.
SEQ ID NO: 2 shows the sequence of the siRNA 5′-CGGAAGGACACACGCUAUA-3′ against TIM-1.
SEQ ID NO: 3 shows the sequence of the siRNA 5′-GCAGAAACCCACCCUACGA-3′ against TIM-1.
SEQ ID NO: 4 shows the sequence of the siRNA 5′-GGUCACGACUACUCCAAUU-3′ against TIM-1.
SEQ ID NO: 5 shows the amino acid sequence of TIM-1 receptor referenced under the GenBank Number AAH13325.1.
SEQ ID NO: 6 shows the nucleic acid sequence of TIM-1 receptor referenced under the NCBI Reference Sequence NM_{—}012206.2.
SEQ ID NO: 7 shows the nucleic acid sequence of TIM-1 receptor referenced under the NCBI Reference Sequence NM_{—}001099414.1.
SEQ ID NO: 8 shows the nucleic acid sequence of TIM-1 receptor referenced under the NCBI Reference Sequence NM_{—}001173393.1.
SEQ ID NO: 9 shows the amino acid sequence of TIM-3 receptor referenced under the GenBank Number AAH20843.1.
SEQ ID NO: 10 shows the amino acid sequence of TIM-3 receptor referenced under the GenBank Number AAH63431.1.
SEQ ID NO: 11 shows the nucleic acid sequence of TIM-3 receptor referenced under the NCBI Reference Sequence NM_{—}032782.4.
SEQ ID NO: 12 shows the amino acid sequence of TIM-4 receptor referenced under the NCBI Reference Sequence NP_{—}612388.2.
SEQ ID NO: 13 shows the amino acid sequence of TIM-4 receptor referenced under the NCBI Reference Sequence NP_{—}001140198.1.
SEQ ID NO: 14 shows the nucleic acid sequence of TIM-4 receptor referenced under the NCBI Reference Sequence NM_{—}138379.2.
SEQ ID NO: 15 shows the nucleic acid sequence of TIM-4 receptor referenced under the NCBI Reference Sequence NM_{—}001146726.1.
SEQ ID NO: 16 shows the amino acid sequence of Annexin 5 referenced under the NCBI Reference Sequence NP_{—}001145.1.
SEQ ID NO: 17 shows the nucleic acid sequence of Annexin 5 referenced under the NCBI Reference Sequence NM_{—}001154.3.
SEQ ID NO: 18 shows the amino acid sequence of TYRO-3 receptor referenced under the NCBI Reference Sequence NP_{—}006284.2.
SEQ ID NO: 19 shows the nucleic acid sequence of TYRO-3 receptor referenced under the NCBI Reference Sequence NM_{—}006293.3.
SEQ ID NO: 20 shows the amino acid sequence of AXL receptor referenced under the NCBI Reference Sequence NP_{—}001690.2.
SEQ ID NO: 21 shows the amino acid sequence of AXL receptor referenced under the NCBI Reference Sequence NP_{—}068713.2.
SEQ ID NO: 22 shows the nucleic acid sequence of AXL receptor referenced under the NCBI Reference Sequence NM_{—}021913.3.
SEQ ID NO: 23 shows the nucleic acid sequence of AXL receptor referenced under the NCBI Reference Sequence NM_{—}001699.4.
SEQ ID NO: 24 shows the amino acid sequence of MER receptor referenced under the NCBI Reference Sequence NP_{—}006334.2.
SEQ ID NO: 25 shows the nucleic acid sequence of MER receptor referenced under the NCBI Reference Sequence NM_{—}006343.2.
SEQ ID NO: 26 shows the amino acid sequence of Gas6 protein referenced under the NCBI Reference Sequence NP_{—}000811.1.
SEQ ID NO: 27 shows the amino acid sequence of Gas6 protein referenced under the NCBI Reference Sequence NP_{—}001137417.1.
SEQ ID NO: 28 shows the amino acid sequence of Gas6 protein referenced under the NCBI Reference Sequence NP_{—}001137418.1.
SEQ ID NO: 29 shows the nucleic acid sequence of Gas6 protein referenced under the NCBI Reference Sequence NM_{—}000820.2.
SEQ ID NO: 30 shows the nucleic acid sequence of Gas6 protein referenced under the NCBI Reference Sequence NM_{—}001143945.1.
SEQ ID NO: 31 shows the nucleic acid sequence of Gas6 protein referenced under the NCBI Reference Sequence NM_{—}001143946.1.
SEQ ID NO: 32 shows the sequence of the siRNA 5′-ACAGCGAGAUUUAUGACUA-3′ against AXL.
SEQ ID NO: 33 shows the sequence of the siRNA 5′-GGUACCGGCUGGCGUAUCA-3′ against AXL.
SEQ ID NO: 34 shows the sequence of the siRNA 5′-GACGAAAUCCUCUAUGUCA-3′ against AXL.
SEQ ID NO: 35 shows the sequence of the siRNA 5′-GAAGGAGACCCGUUAUGGA-3′ against AXL.
SEQ ID NO: 36 shows the sequence of the variant Gas6ΔGla protein.
SEQ ID NO: 37 shows the sequence of an external primer for TYRO-3 cloning.
SEQ ID NO: 38 shows the sequence of an internal primer for TYRO-3 cloning.
SEQ ID NO: 39 shows the sequence of an internal primer for TYRO-3 cloning.
SEQ ID NO: 40 shows the sequence of an external primer for TYRO-3 cloning.
SEQ ID NO: 41 shows the sequence of a primer for AXL cloning.
SEQ ID NO: 42 shows the sequence of a primer for AXL cloning.
SEQ ID NO: 43 shows the sequence of a primer for TIM-1 ectodomain amplification.
SEQ ID NO: 44 shows the sequence of a primer for TIM-1 ectodomain amplification.
SEQ ID NO: 45 shows the sequence of a primer for TIM-4 ectodomain amplification.
SEQ ID NO: 46 shows the sequence of a primer for TIM-4 ectodomain amplification.
SEQ ID NO: 47 shows the amino acid sequence of TIM-1 receptor referenced under the UniProt Number Q96D42.

### FIGURES

FIG. 1. TIM receptors mediate DV infection. The 293T cells, were challenged with DV2-JAM at the indicated multiplicities of infection (MOI). Infection levels were assessed two days later by flow cytometry using the antiNS1 mAb. Data are means±SD of at least three independent experiments.

FIG. 2. TIM receptors mediate DV infection. TIM receptors are used by the four DV serotypes. Cells were infected by DV1-TVP, DV3-PAH881 and DV4-1086. Infection was assessed two days later by flow cytometry using the anti-PrM 2H2 mAb. Data are means±SD of at least three independent experiments.

FIG. 3. TIM receptors mediate DV infection. TIM receptors enhance infection by the laboratory-adapted DV2 New Guinea C (NGC) and 16681 strains. Data are means±SD of at least three independent experiments.

FIG. 4. TIM-1 and TIM-4 molecules bind to DV. Western blot analysis of DV2-JAM preincubated with control Fc, NKG2D-Fc, TIM1-Fc, or TIM-4-Fc bound to protein A-agarose beads. Pulled-down virus was detected using the 4G2 anti-DV E protein mAb. Data are means±SD of at least three independent experiments. **p<0.001, ***p<0.0001.

FIG. 5. TIM-1 and TIM-4 molecules bind to DV. Interaction of DV with soluble TIM-1-Fc. Control Fc, NKG2D-Fc or TIM-1-Fc were coated on plastic in 96-well plates and incubated with DV2-JAM particles for 1 hour at 4° C. Bound virus was detected using the biotinylated 4G2 mAb and HRP-conjugated anti-mouse IgG. Data are means±SD of at least three independent experiments. **p<0.001, ***p<0.0001.

FIG. 6. TIM-1 and TIM-4 molecules bind to DV. PtdSer are associated with DV virions. DV2 particles were coated on well plates and incubated with the anti-PtdSer 11-16 mAb. Data are means±SD of at least three independent experiments. **p<0.001, *** p<0.0001.

FIG. 7. TIM-1 and TIM-4 molecules bind to DV. TIM-mediated DV infection is PtdSer-dependent. DV2-JAM (MOI=5) preincubated with Annexin V (ANX5; 25 pg/ml) was used to infect the indicated cells. Levels of infected cells were quantified 48 hours later by flow cytometry and normalized relative to infection without Annexin V. Data are means±SD of at least three independent experiments. **p<0.001, ***p<0.0001.

FIG. 8. TIM molecules mutated in the PtdSer binding domain do not mediate DV infection. Transfected cells were infected with DV2-JAM. The percentages of infected cells (at day 2) are shown. Data are means±SD of at least three independent experiments. **p<0.001, ***p<0.0001.

FIG. 9. Endogenous TIM-1 and AXL molecules mediate DV infection. Huh7.5.1 cells were infected with the indicated DV strains or HSV-1 in the presence of anti-TIM-1, anti-AXL or control IgG. The levels of infected were quantified 24 h later by flow cytometry and normalized to infection in presence of control IgG. Data are means±SD of at least three independent experiments. **p<0.001, ***p<0.0001.

FIG. 10. Endogenous TIM-1 and AXL molecules mediate DV infection. A549 cells were infected with the indicated DV strains or HSV-1 in the presence of anti-TIM-1, anti-AXL or control IgG. The levels of infected were quantified 24 h later by flow cytometry and normalized to infection in presence of control IgG. Data are means±SD of at least three independent experiments. **p<0.001, ***p<0.0001.

FIG. 11. Endogenous TIM-1 and AXL molecules mediate DV infection. Representative immunofluorescence analysis of A549 infected with DV2-JAM in the presence of the indicated Ab. Green anti-PrM 2H2, Blue DAPI. Scale bar: 100 urn. Data are means±SD of at least three independent experiments. **p<0.001, ***p<0.0001.

FIG. 12. Endogenous TIM-1 and AXL mediate DV infection. A549 cells were infected with DV3-PAH881 (MoI=10). Prior infection cells were incubated with indicated combination of anti-TIM-1 and anti-AXL polyclonal antibodies. Infection levels were quantified 24 hours later by flow cytometry and normalized to infection level in the presence of IgG control antibody. Means±SD from three independent experiments in duplicate are shown.

FIG. 13. Effect of TIM-1 and AXL silencing on DV infection. A549 cells were transfected by the indicated siRNA, and TIM-1 and AXL expression was assessed by flow cytometry after two days, at the time of infection. Cells were infected with DV2-JAM (MOI=2) or HSV-1 (MOI=0.8). The levels of infected cells were quantified 24 h later by flow cytometry and normalized to infection in non-targeting (siNT) siRNAtransfected cells. Data are means SD of at least three independent experiments. **p<0.001, ***p<0.0001.

FIG. 14. A549 cells were infected with DV-2 JAM or HSV-1 pre-incubated with different concentrations of ANX5. Infected cell percentages were quantified 24 hours later by flow cytometry. Data are means±SD of at least three independent experiments. **p<0.001, ***p<0.0001.

FIG. 15. Schematic model of direct phosphatidylserine-TIM receptor binding of DV. The phosphatidylserine interacts directly with TIM receptors, which consequently either trigger a signal transduction cascade that results in innate immunity inhibition or mobilization of endocytosis effectors that enhance virus internalization.

FIG. 16. TIM receptors mediate flavivirus infection. TIM receptors are used by DV2-JAM, West Nile Virus and Yellow Fever Virus. Parental and 293T cells expressing TIM receptors were infected by DV2-JAM, WNV (Israeli IS_{—}98-STI strain), Yellow Fever Virus vaccine strain (YFV-17D) and Herpes Simplex Virus 1 (HSV-1). Viral infection was quantified two days later by flow cytometry using specific Abs. Data are means±SEM of at least three independent experiments.

FIG. 17. TYRO3 and AXL enhance infection by DV and by other flaviviruses. Parental and TYRO3- and AXL-expressing 293T were challenged with DV2-Jam, WNV, YFV-17D and HSV-1. Infection was assessed 24 hours later by flow cytometry. Data are represented as mean±SEM from three independent experiments in duplicate.

FIG. 18. TIM-1 and TIM-4 ectopic expression enhance infection by Chikungunya. TIM-1, TIM-4 expressing 293T cells and parental 293T cells were infected with Chikungunya (Chick). Infection was quantified 48 hours later by flow cytometry, using a mouse monoclonal antibody against the E2 envelope glycoprotein (3E4).

FIG. 19. TYRO3 and AXL ectopic expression enhance infection by Chikungunya. TYRO, AXL expressing 293T cells and parental 293T cells were infected with Chikungunya (Chik). Infection was quantified 48 hours later by flow cytometry, using a mouse monoclonal antibody against the E2 envelope glycoprotein (3E4).

### EXAMPLE

### Material and Methods

cDNA library screening

For the cDNA screen, 1728 genes encoding putative cellular receptors were selected based on bioinformatics from an arrayed full-length cDNA library 33. In the first round of screening, 216 pools of 8 cDNAs were transfected into 293T cells using Lipofectamine LTX. Transfected 293T cells were then incubated with DV2-JAM primary strain (MOI=2) for 48 hours and infection was scored by FACS using the 2H2 mAb that recognizes the DV prM protein. Pools of cDNA that rendered 293T cells positive for prM protein intracellular staining entered the second round of screening, in which single cDNA composing each pool were individually tested.

### Viruses and Cells

The DV-1-TVP strain, DV2-JAM strain (Jamaica), DV2-New Guinea C strain, DV2-16881 strain, DV3-PAH881 strain (Thailand) and DV4-1086 strain were propagated in mosquito (Aedes pseudoscutellaris) AP61 cell monolayers after having undergone limited cell passages. Of note, DV produced in mammalian cells gave similar results than viruses originating from insect cells. Virus titers were assessed by flow cytometry analysis (FACS) on C6/36 cells and were expressed as FACS infectious units (FIU). HEK 293T, A549, VERO, and Huh7 5.1 cells (a gift of C. Rice, New York, USA) were maintained in DMEM supplemented with 10% FBS, 1% penicillin/streptomycin. Human primary astrocytes and epithelial cells were purchased from LONZA and cultured according to the manufactured conditions.

DV2-JAM (Jamaica) and WNV (Israeli IS-98-STI strain was propagated in mosquito (Aedes pseudoscutellaris) AP61 cell monolayers as described above. YFV (strain YFV D17) was grown and titrated on Vero cells. HSV-1 (F) was propagated and titrated on Vero cells as described as described elsewhere (Taddeo et al. 2004). Chikungunya (strain CHIKV-21) was grown in insect cells C6/36.

### Proteins and Antibodies

Recombinant murine Gash lacking the N-terminal Gla domain (rmGas6ΔGla), recombinant human IgG1-Fc, TYRO3-Fc, AXL-Fc, DC-SIGN-Fc, TIM-3-Fc and NKG2D-Fc were from R&D systems. Antibodies were as follows: mouse monoclonal (mAb) anti-human TIM-1 (clone 219211), anti-human TYRO3 (clone 96201), anti-human AXL (clone 108724), IgG2b isotype (MAB004), IgG1 isotype (clone 11711), anti-human DC-SIGN PE-conjugated (clone Clone 120507), IgG2B PE-conjugated isotype (clone 133303), goat polyclonal (pAb) anti-human TIM-1 (AF1750), anti-human TIM-4 (AF2929), anti-human Tyro3 (AF859), anti-human AXL (AF154) were from R&D systems. Mouse monoclonal anti-human phosphatidylserine (1H6) was purchased from Millipore. Polyclonal rabbit anti-human IgG-HRP was from DakoCytomation and the Donkey anti-goat IgG-HRP was from Santa Cruz biotechnologies.

### Plasmid Constructs

Tim-1 and Tim-4 gene open reading frames (ORF) were amplified from cDNAs respectively purchased from Life Technologies and Origene. Tim-3 ORF was amplified from the cDNA clone identified in the screen. All TIM ORFs were cloned into pCDNA3.1 and pTRIP vectors using BamHI and XhoI restriction sites.

Tyro3 and AxI gene ORFs were amplified from the cDNA clones identified in the screen and cloned in the pTRIP vector. To create pTRIP-Tyro3, the ORF was amplified and the internal BamHI site was simultaneously removed using site-specific silent mutagenesis (T1155C) by the overlapping extension method. A first fragment was amplified with the external primer 5′ CGGGATCCCGC ATG GCG CTG AGG CGG AGC ATGG (SEQ ID NO: 37, start codon in bold; restriction endonucleases site underlined) and the internal primer 5′ GTCCTITTGGGGGTCCCAGCCTGTCAAATTGGC (SEQ ID NO: 38, mutated nucleotide underlined). The second fragment was amplified with the internal primer 5′ GCCAATTTGACAGGCTGGGACCCCCAAAAGGAC (SEQ ID NO: 39, mutated nucleotide underlined) and the external primer 5′ CCGCTCGAGCGG CTA ACA GCT ACT GTG TGG CAG TAG CCC (SEQ ID NO: 40, stop codon bold; restriction endonuclease sites underlined). Following purification, both fragments were mixed and full length ORF was finally amplified with the two external primers. This product was cloned as a BamHI and XhoI digested fragment into a likewise digested pTRIP plasmid. AxI ORF was amplified with oligos 5′ CGGGATCCCGC ATG GCG TGG CGG TGC CCC (SEQ ID NO: 41) and 5′ CCGCTCGAGCGG TCA GGC ACC ATC CTC CTG CCC (SEQ ID NO: 42). This fragment was cloned as a BamHI/XhoI fragment into the likewise digested pTRIP plasmid. Alanine, substitution mutants of Tim-1, Tim-4 and AxI, were generated using the Quick Change Site Directed Mutagenesis Kit (Agilent).

### Establishment of Stable Cell Lines Overexpressing TIM-1, TIM-4, TYRO3 and AXL

Pseudoviruses were generated according to conventional calcium-phosphate transfection protocol by co-transfection of pTRIP constructs with plasmids encoding HIV gag-pol and vesicular stomatis virus envelope G (VSVg) protein in 293T cells. Two days later, supernatants were harvested, cleared by low-speed centrifugation and pseudoparticles were concentrated by ultracentrifugation. Pellets were resuspended in THE buffer (Tris 50 mM, NaCl 100 mM and EDTA 0.5 mM), aliquoted and stored at −80° C. 293T cells (1.5×10⁵) were transduced with pseudoviruses carrying the desired ORF. Cell populations with high cell surface expression of TIM-1, TIM-4, TYRO3 and AXL were sorted with a BD FACSAria II (Becton Dickinson) with FACSDiva 6.1.2 software (Becton Dickinson).

Production of TIM-Fcs and rGas6

TIM-1 and TIM-4 fusion proteins with human IgG1 Fc were generated as follows. TIM-1 ectodomain (residues 21-290) was amplified with the 5′ ATCGGAGATATCT GTA AAG GTT GGT GGA GAG GCA GGT CC (SEQ ID NO: 43) and the 3′ TCTGGAAGATCTTCC TTT AGT GGT ATT GGC CGT CAG (SEQ ID NO: 44) primers. TIM-4 ectodomain (residues 25-314) was amplified with the 5′ ATCGGAGATATCA GAG ACT GTT GTG ACG GAG GTT TTG GG (SEQ ID NO: 45) and 3′ TCTGGAAGATCTTTG GGA GAT GGG CAT TIC ATT CTTC (SEQ ID NO: 46) primers. Both PCR products were cloned in pFUSE-hIgG1-Fc2 (Invivogen) using EcoRV and BgIII restriction sites (first and last TIM codons in bold; restriction endonuclease sites underlined). TIM-1- and TIM-4-Fc fusion expressing vectors were transfected in 293T cells in Iscove's Modified Dulbecco's Medium supplemented with 10% FBS and cultured after transfection in OPTIPRO-SFM (Life Technologies). Both media were supplemented with P/S and L-glutamine. Four days post-transfection, supernatants were harvested, cleared by centrifugation and concentrated through Amicon 50K MWCO (Millipore). TIM-Fcs were purified on a Protein A column and concentrated/desalted through 30K MWCO PES filter units (Pierce). Proteins were stored in phosphate-buffered saline (PBS), 0.02% NaN₃ and subsequently aliquoted at −80° C. Proteins were quantified using 280 nm absorbance and their purity was assessed in reducing conditions with Coomassie Blue staining (R250) of samples run in SDS-PAGE conditions.

A mammalian expression vector was engineered to encode full length mouse Gas6 followed by a C-terminal, TEV cleavable His₆-tag. The construct was transfected into 293T cells, and cells stably expressing the construct were selected in Dulbecco's Modified Eagle Medium supplemented with 10% FBS, 0.25 mg/mL G418, and 100 μg/mL hygromycin. For expression studies, cells were grown in serum free medium supplemented with 10 μM Vitamin K2, and conditioned medium was collected after 72 hours. Secreted Gas6 was isolated using affinity chromatography with Ni-NTA beads followed by additional purification on a Hi Trap 0 Fast Flow ion exchange column. The protein was eluted in 20 mM Tris, pH 8 with 0-1 M NaCl gradient, and was subsequently aliquoted and flash-frozen in liquid N₂.

### ELISA Binding

For detecting direct interactions between TIM-Fc and DV, Fc fused proteins were first coated (duplicates, 400 ng/well) in Tris-Buffered Saline (TBS) supplemented with 10 mM CaCl₂ on 96-well Maxisorp NUNC-IMMUNO plates (NUNC), overnight at 4° C. Wells were washed with TBS 10 mM CaCl₂ and saturated for 2 hours at 37° C. with TBS 10 mM CaCl₂, 2% BSA. After extensive washing with TBS 10 mM CaCl₂, 0.05% Tween, DV particles (5.10⁶ FACS infectious unit (FIU)/well) were added and incubated for 2 hours at 4° C. Bound particles were detected with the biotinylated 4G2 antibody (1 μg/ml) and Horseradish peroxydase (HRP)-conjugated Streptavidine (R&D systems).

For Gas6 bridging experiments, DV particles (10⁷ FIU) were coated at 4° C. overnight in duplicates. Following blocking with 2% BSA in PBS CaCl₂/MgCl₂ at 37° C. for 1 hour, wells were incubated with rGas6 proteins (2 μg/ml) and Fc-chimera proteins (2 μg/ml) for 1 hour at 37° C. in TBS 10 mM CaCl₂, 0.05% Tween. Wells were extensively washed and bound Fc-chimeras were detected with HRP-conjugated rabbit anti-human IgG antibody. For Gas6 binding experiments, DV particles (10⁷ FIU) or PtdSer (3-sn-Phosphatidyl-L-serine from bovine brain) were coated overnight in duplicates. Wells were incubated with rGas6 proteins (2 μg/ml) and extensively washed. Bound Gas6 proteins were labeled with a goat anti-Gash polyclonal antibody and detected with a HRP-conjugated donkey anti-goat IgG antibody (Santa Cruz Biotechnology).

PtdSer was detected on coated DV particles (10⁷ FIU) using anti-PtdSer 1H6 mAb (10 μg/ml) and a HRP-conjugated rabbit anti-mouse IgG antibody in PBS BSA 2%.

### Virus Pull-Down

DV particles (10⁷ FIU) were incubated overnight at 4° C. with 2 μg of Fc-chimera proteins in TBS, 10 mM CaCl2. BSA saturated Protein G Sepharose beads (GE Healthcare) were added and incubated for 4 hours at 4° C. Beads were washed 4 times with TBS, 10 mM CaCl₂, 0.05% Tween, and bound materiel was resolved in 1× Laemmli buffer in non-reducing conditions. Nitrocellulose-bound E envelope glycoprotein was detected with the 4G2 mAb and HRP-conjugated rabbit anti-mouse IgG antibody (Sigma-Aldrich).

### Cell Binding Assay

293T cells expressing TIM-1, TIM-4, TYRO3, AXL or DC-SIGN (4×10⁵) were incubated with the indicated MOI of DV for 90 minutes at 4° C. in binding buffer (DMEM, NaN3 0.05%) containing either 2% BSA or 5% FBS. Cells were incubated with 100 U heparin for 30 min at room temperature, before incubation with the virus. The cells were washed twice with cold binding buffer, once with serum-free cold DMEM, and fixed in PBS-PFA 2% at 4° C. for 20 minutes. Cell surface absorbed DV particles were stained with the anti-panflavivirus envelope 4G2 antibody (5 μg/ml) and analyzed by flow cytometry. For bridging assays, cells were simultaneously incubated with virus and rGas6 (10 μg/ml).

### Flow Cytometry Analysis

Flow cytometry analysis was performed by following a conventional protocol in the presence of 0.02% NaN3 and 5% FBS in cold PBS. For infection assays, infected cells were fixed with PBS plus 2% (v/v) paraformaldehyde (PFA), permeabilized with 0.5% (w/v) saponin, followed by staining with mouse 2H2 mAb detecting DV prM (2 μg/ml), or mouse NS1 mAb detecting the nonstructural protein-1 (1 μg/ml). HSV-1 infection was detected with anti-ICP4 mouse mAb (clone 10F1, 0.3 μg/ml; Santa Cruz Biotechnology). WNV, YFV and Chikungunya infection were detected with the antibody anti-protein E (4G2) and a mouse monoclonal antibody against the E2 envelope glycoprotein (3E4). After 45 minutes, primary antibodies were labeled with a polyclonal goat anti-mouse immunoglobulin/RPE (DakoCytomation). Finally, infected cells percentages were assessed by flow cytometry on a LSR with CellQuest software (Becton Dickinson). Data were analyzed by using the FlowJo software (Tree Star).

### Immunofluorescence Assay

Cells were cultured on Lab-Tek II-CC² Chamber Slide (Nunc, Roskilde, Denmark) and incubated with indicated amounts of DV2-JAM for 24 or 48 hours. After incubation, cells were fixed with PBS-PFA 4% (v/v), permeabilized with 0.05% (w/v) saponin in PBS, and incubated 10 min in PBS glycine 0.1 M, followed by incubation with blocking buffer before immunostaining of DV prM protein (2H2, 5 μg/ml). Slides were mounted with Moviol containing 4,6-diamidino-2-phenylindole (DAPI) for nuclei staining (Life Technologies).

### Inhibition of Infection Assay

For inhibition experiments, cells grown on 24-well plates, were incubated for 30 minutes prior to infection with media containing the indicated quantities of anti-TIM and/or anti-TAM antibodies. Identical concentrations of normal goat IgG were used as respective mock control. After 3 hours incubation with DV or HSV in the presence of inhibitors, medium was changed and cells were incubated with culture medium. Infection was quantified by FACS as indicated above.

### RNA Interference

A549 cells and primary astrocytes were transiently transfected using the Lipofectamine RNAiMax protocol (Life Technologies) with 10 nM final siRNAs. After 48 hours, cells were infected at the indicated MOI, and infected cells percentages were quantified 24 hours post-infection by flow cytometry. Pools of siRNAs (ON-TARGETpIus SMARTpool) used in this study were from Dharmacon: TIM-1 (L019856-00), AXL (L-003104-00). Non-targeting negative control (NT) was used as control.

### Statistical Analyses

Graphical representation and statistical analyses were performed using Prism5 software (GraphPad Software). Unless otherwise stated, results are shown as means+/−standard deviation (SD) from 3 independent experiments. Differences were tested for statistical significance using the paired two-tailed t test.

### Results and Discussion

To identify new DV entry factors, 1728 plasma membrane proteins were screened for their ability to render the poorly susceptible 293T cell line sensitive to primary mosquito-derived DV2-JAM strain. This screen identified L-SIGN, confirming the validity of the approach, but also T-cell immunoglobulin domain and mucin domain (TIM)-3, TYRO3 and AXL as novel potential DV receptors. These belong to two distinct families of transmembrane receptors that bind directly (TIMs) or indirectly (TAMs) phosphatidylserine (PtdSer), an ‘eat me’ signal that promotes the engulfment of apoptotic cells. The role of these receptors and of PtdSer during DV infection was then characterized.

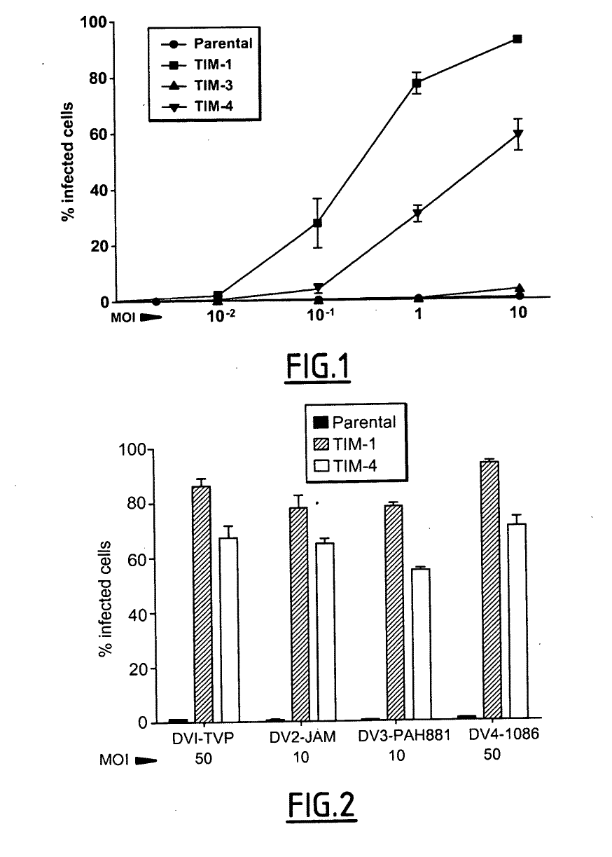

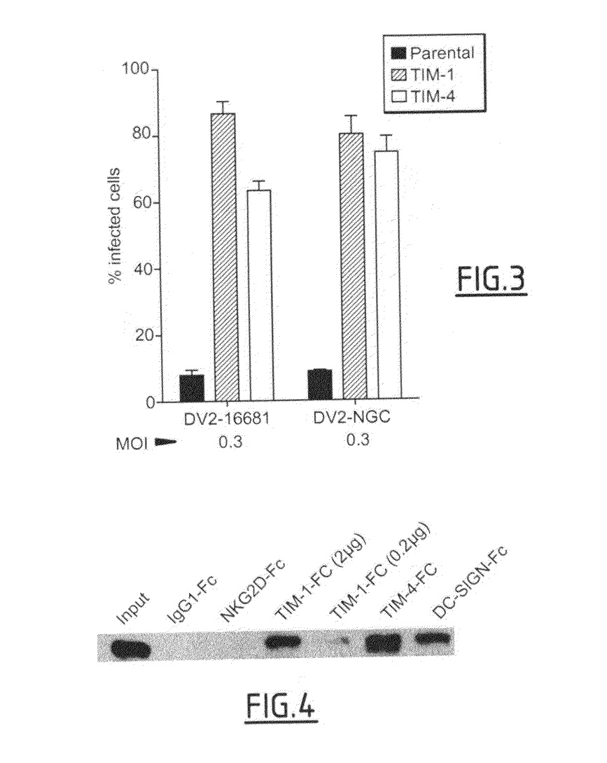

TIM-3, along with TIM-1 and TIM-4, modulates immune tolerance, likely through the clearance of dead cells. Moreover, the Hepatitis A virus and filoviruses use TIM-1 as a receptor. To examine whether TIM receptors enhance DV infection, 293T cells stably expressing TIM-1 and TIM-4 or TIM-3 were generated and challenged with DV2-JAM. Parental cells, which do not express TIM molecules, were minimally infected by the virus (FIG. 1). TIM-3 expression resulted in a modest increase of the percentage of infected cells (FIG. 1). Strikingly, TIM-1 or TIM-4 expression potentiated infection up to 500-fold (FIG. 1). Of note, infection was assessed by measuring newly synthesized NS1 proteins, indicating that TIMs mediate productive DV infection. Enhancement of DV infection did not occur in cells expressing BAI1, another PtdSer receptor. TIM-1 or TIM-4 also mediated efficient infection by the three other DV serotypes (FIG. 2). The laboratory-adapted DV2 New Guinea C (NGC) and 16681 strains infected parental 293T cells, suggesting that some isolates may use other(s) receptor(s) (FIG. 3). However, DV2 NGC or 16681 infection was also strongly enhanced by TIM-1 or TIM-4 (FIG. 3). Together, these data indicate the PtdSer receptors TIM-1 and TIM-4, and to a lesser extent TIM-3, are new cellular factors promoting DV infection.

Whether DV virions bind to TIM proteins was examined by conducting a pull-down assay with soluble TIM-Fc (the extracellular region of TIM fused to immunoglobulin Fc). DV-2 particles were incubated with TIM-1-Fc or TIM-4-Fc, or with DC-SIGN-Fc as a positive control. Precipitated virus was analyzed by Western blotting. DV bound to TIM-1, TIM-4 and DC-SIGN constructs, and not to NKG2D-Fc or IgG1-Fc negative control constructs (FIG. 4). This was confirmed by ELISA using TIM-1-Fc coated wells (FIG. 5). Moreover, DV, efficiently attached to 293T-TIM-1 and 293T-TIM-4 but not to control cells. Together, these results show that TIM-1 and TIM-4 bind DV and mediate virus attachment to target cells.

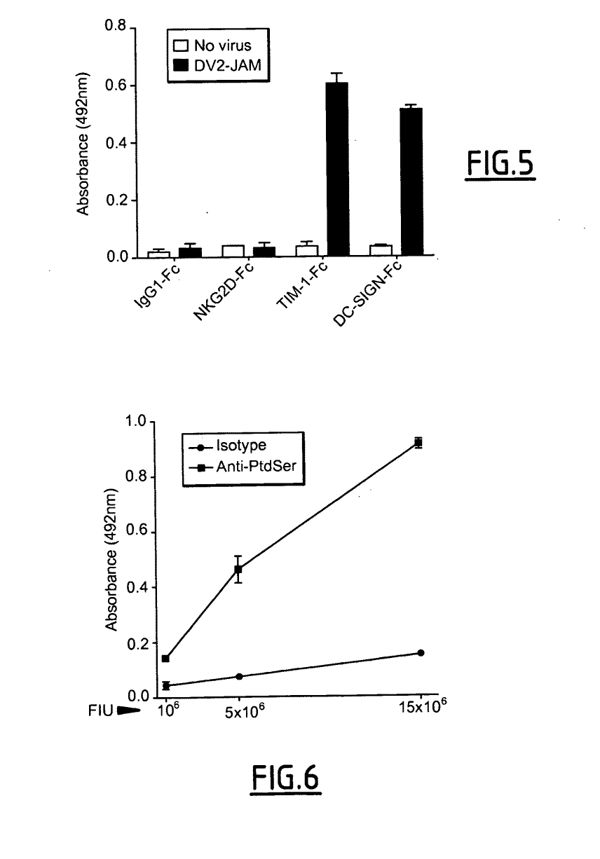

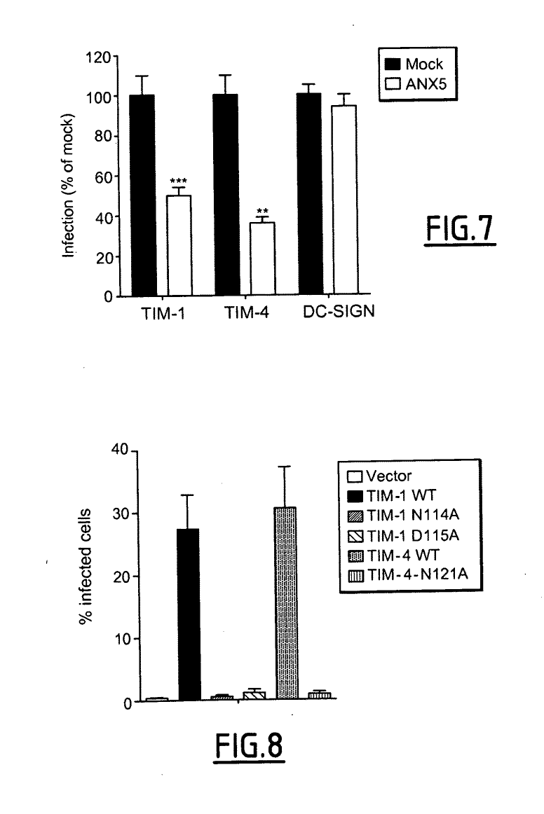

TIM-1 and TIM-4 recognize PtdSer on apoptotic cell bodies. It was further examined if TIM-mediated DV infection depended on PtdSer. An anti-PtdSer monoclonal Ab (mAb), but not its isotype control, bound in a dose-dependent manner to DV-coated ELISA plates (FIG. 6), indicating that PtdSer is associated with DV particles. DV-2 was then preincubated with annexin V (ANX5), a well-documented PtdSer-binding protein. ANX5 inhibited infection of 293T-TIM-1 and 293T-TIM-4 but not of 293T-DCSIGN cells (FIG. 7). Structural studies of TIM have shown that PtdSer binds a cavity termed the metal ion dependent ligand binding site (MILIBS). Mutants of this cavity (TIM-1 N114A or D115A, TIM-4 N121A) were designed, which no longer mediated DV-2 infection even though they were correctly expressed at the cell surface (FIG. 8). Therefore, PtdSer molecules are associated with DV virions and are required for TIM-mediated DV infection. TYRO3 and AXL belong to the TAM family, a group of three receptor protein tyrosine kinases essential for clearance of apoptotic cells. TAM ligands, Gas6 and ProS, play a key role in this process. Via their N-terminal Gla domain, they recognize the PtdSer expressed on apoptotic cells, and bridge these cells to a TAM receptor on the surface of phagocytes. TAM receptors have been shown to promote infection by the Ebola and Lassa viruses and Gas6 was found to enhance infection by lentiviral vectors or vaccinia virus via bridging virus membrane PtdSer to AXL.

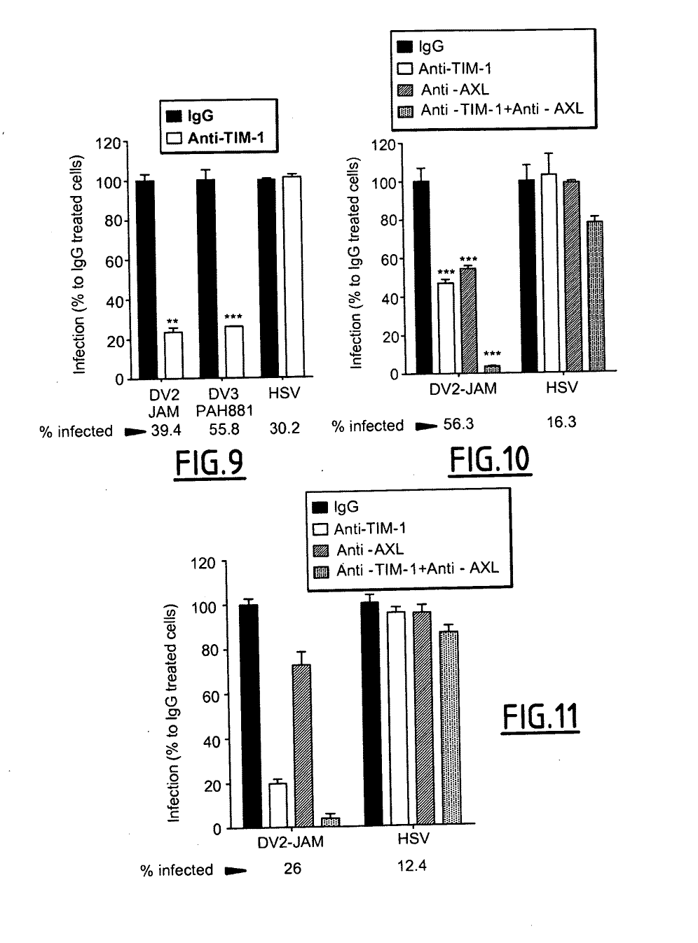

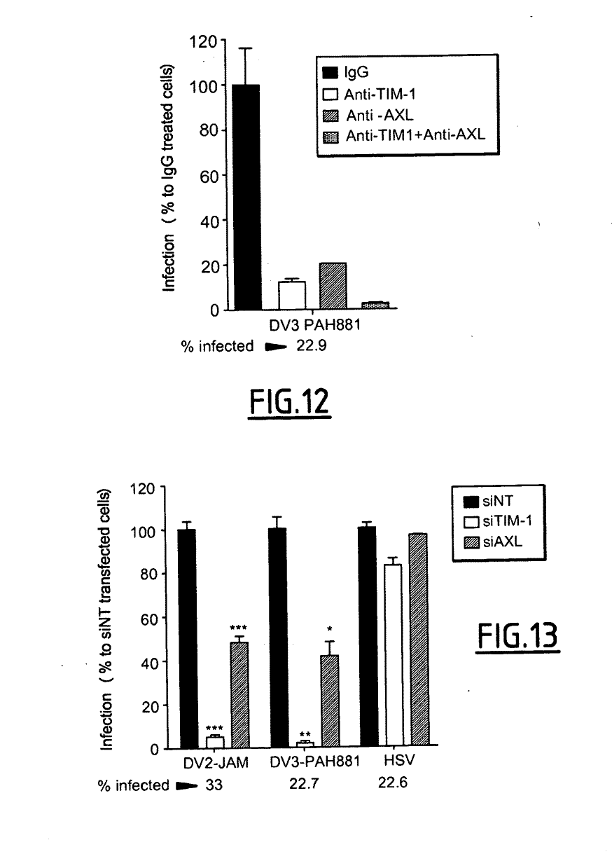

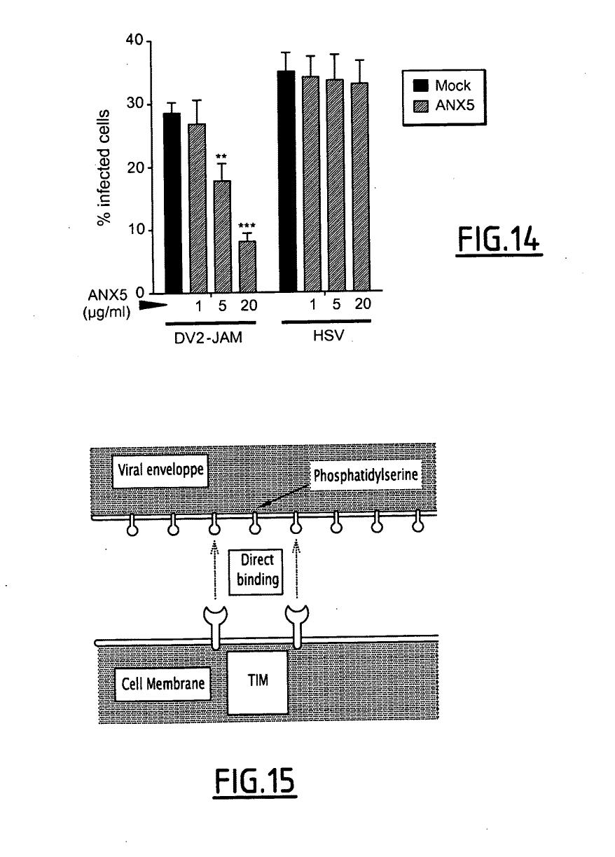

TIM and TAM respective roles in cells naturally expressing these receptors were next investigated. At least one of the four molecules (TIM-1, TIM-3, TYRO3, AXL) was detected in a panel of DV-sensitive cell lines. The Huh7 5.1 cell line expresses only TIM-1. An anti-TIM-1 Ab inhibited DV2 infection but not Herpes Simplex Virus (HSV-1) infection (FIG. 9). The A549 cell line expresses both TIM-1 and AXL. DV2 infection was partly reduced with an anti-TIM-1 or anti-AXL Ab administrated alone, while the two Ab in combination fully inhibited DV2 (FIGS. 10 and 11), DV3 (FIG. 12) but not HSV-1 infection. Similar results were obtained in Vero cells that express TIM-1 and AXL. TIM-1 or AXL was then silenced by RNA interference in A549 cells (FIG. 13). DV infection was reduced in AXL-silenced cells and almost totally inhibited in TIM-1 silenced cells. Notably, as for TIM- and TAM-293T-transfected cells, ANX5 blocked DV infection of A549 cells (FIG. 14). Altogether, these results show that TIM and TAM receptors may naturally cooperate to promote DV infection and that PtdSer is mediating infection in cells endogenously expressing the receptors.

Epithelial cells and astrocytes are DV targets in vivo. Primary kidney epithelial cells and astrocytes express AXL and not TYRO3, TIM-1 or TIM-4. DV infection was significantly reduced by an anti-AXL Ab in both cell types. Silencing AXL in astrocytes also significantly decreased DV2-JAM infection. Therefore, as demonstrated for AXL, the PtdSer receptors identified in our screening are involved in the infection of human primary cells, an observation that should be relevant for DV pathogenesis.

This report identifies TIM and TAM receptors of PtdSer as novel cellular factors mediating DV binding to, and infection of target cells (FIG. 15). PtdSer is an “eat me” signal for the recognition and clearance of apoptotic cells by phagocytes. Thus, DV use an “apoptotic mimicry” strategy to infect cells. By utilizing at least four different PtdSer receptors, alone or in combination, DV may gain access to multiple cell types, consistent with the wide viral tropism observed in DV-infected patients.

DV membrane is derived by budding into the ER, that contains PtdSer in the luminal side, suggesting an obvious mechanism through which PtdSer becomes incorporated into virions. However, structural studies indicate that the membrane is not readily exposed in mature particles, in which it would be hidden beneath a protective icosahedral shell formed by the E protein. It is plausible that TIM and TAM molecules or other receptors may display weak interactions with the E protein that trigger opening of the icosahedral shell, leading to exposure of viral membrane, as recently suggested by studies with Ab complexes. Also, recent reports indicate an important degree of heterogeneity in this glycoprotein shell, which displays a mixture of immature and mature surfaces. The immature-like regions could expose membrane patches, such that PtdSer would be accessible to interact with the TIM and TAM receptors.

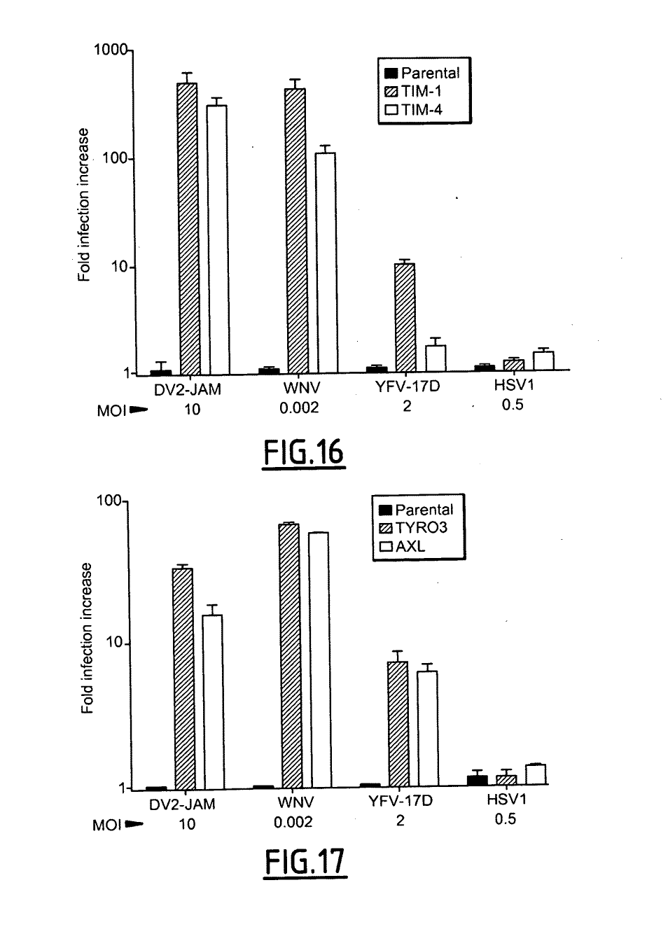

To determine whether TIM and TAM receptors mediate infection by other viral species, TIM-1- and TIM-4-expressing cells were challenged with DV2-Jam West Nile virus (WNV), Yellow Fever Virus vaccine strain (YFV-17D), and Herpes Simplex Virus 1 (HSV-1). Viral infection was quantified by flow cytometry using specific Antibodies (FIG. 16). The data show that TIM-1 and TIM-4 massively enhanced WNV infection, slightly upregulated sensitivity to YFV-17D, but had no effect on HSV-1. Similar results were obtained for TYRO3- and AXL-expressing cells (FIG. 17). Together, these data indicate the PtdSer receptors TIM and TAM are both cellular factors promoting flavivirus infection.

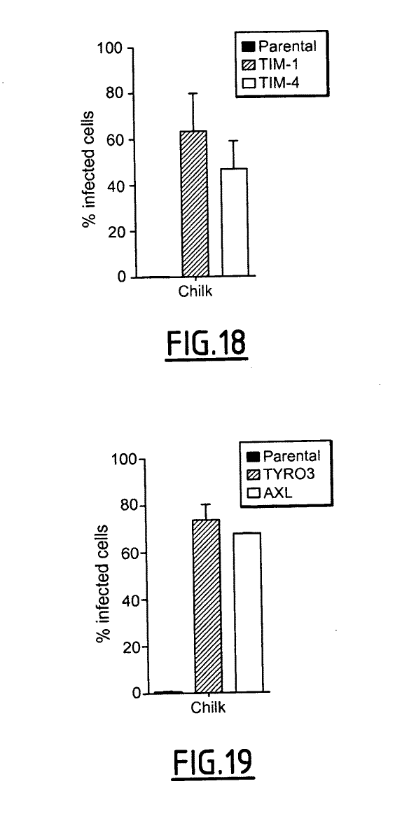

Furthermore, it was of interest if this mechanism represents a general mechanism exploited by viruses that express or incorporate PtdSer in their membrane. Parental 293T cells, TIM-1 and TIM-4 expressing 293T cells were infected with Chikungunya (Chick). Infection was quantified 48 hours later by flow cytometry using a mouse monoclonal antibody against the E2 envelope glycoprotein (3E4). The results (FIG. 18) show that TIM-1 and TIM-4 massively enhance Chikungunya infection. Similar results were obtained for TYRO3 and AXL expressing cells, their ectopic expression enhances as well Chikungunya infection (FIG. 19).

These data show that TIM and TAM facilitation of viral infection represents a general mechanism exploited by viruses that express or incorporate PtdSer in their membrane for optimal infection.

1. A method for preventing of treating a viral infection comprising administering to an individual in need thereof a therapeutically effective amount of an inhibitor of an interaction between phosphatidylserine and a TIM receptor, wherein said inhibitor is:
(i) TIM receptor inhibitor, and/or
(iii) a phosphatidylserine binding protein

2. The method according to claim 1(i), wherein said TIM receptor is TIM-1, TIM-3 or TIM-4.

3. The method according to claim 1, wherein said TIM receptor inhibitor is an anti-TIM receptor antibody, an antisense nucleic acid, a mimetic or a variant TIM receptor.

4. The method according to claim 1, wherein said phosphatidylserine binding protein is an anti-phosphatidylserine antibody or Annexin 5.

5. The method according to claim 3, wherein said TIM receptor inhibitor is a siRNA of sequence SEQ ID NO: 1, 2, 3, or 4.

6. The method according to claim 1, wherein said virus is a phosphatidylserine harboring virus.

7. The method according to claim 6, wherein said phosphatidylserine harboring virus is an Alphavirus or a Flavivirus.

8. The method according to claim 7, wherein said Alphavirus is Chikungunya virus.

9. The method according to claim 7, wherein said Flavivirus is a West-Nile Virus, Yellow Fever Virus or Dengue Fever Virus.

10. A method according to claim 1, wherein said inhibitor is for administration in combination with at least one other antiviral compound, either sequentially or simultaneously.

11. A method according to claim 10, wherein said other antiviral compound is an inhibitor of an interaction of phosphatidylserine and a TAM receptor.

12. A method according to claim 11, wherein said inhibitor of an interaction of phosphatidylserine and a TAM receptor is:
(i) a TAM receptor inhibitor, and/or
(ii) a Gas6 inhibitor.

13. A method according to claim 1, wherein said inhibitor is formulated in a pharmaceutically acceptable composition.

14. A pharmaceutical composition comprising an inhibitor as defined in claim 1 and additionally at least one other antiviral compound.

15. A pharmaceutical composition according to claim 14, wherein said at least one other antiviral compound is an inhibitor of an interaction of phosphatidylserine and a TAM receptor.

16. A pharmaceutical composition according to claim 15, wherein said inhibitor of an interaction of phosphatidylserine and a TAM receptor is:
(i) a TAM receptor inhibitor, and/or
(ii) a Gas6 inhibitor.

17. A method of inhibiting entry of a phosphatidylserine harboring virus into a cell comprising exposing said cell to an inhibitor as defined in claim 1.

18. (canceled)


SEQUENCE LISTING (37 sequences)

SEQ ID NO: 1 - RNA, 19 nt, Artificial Sequence
  Synthetic siRNA against AXL receptor
acagcgagauuuaugacua

SEQ ID NO: 2 - RNA, 19 nt, Artificial Sequence
  Synthetic siRNA against AXL receptor
gguaccggcuggcguauca

SEQ ID NO: 3 - RNA, 19 nt, Artificial Sequence
  Synthetic siRNA against AXL receptor
gacgaaauccucuauguca

SEQ ID NO: 4 - RNA, 19 nt, Artificial Sequence
  Synthetic siRNA against AXL receptor
gaaggagacccguuaugga

SEQ ID NO: 5 - protein, 890 aa, Homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 6 - DNA, 4001 bp, Homo sapiens
agtggaaggagcgcggtggcgcgggagcggccccggggaccccgcgctgctgacggcggcgaccgcggccggaggcgggcgcgggtctcggaggcggtcgcctcagcaccgccccacgggcggccccagcccctcccgcagccctcctccctcccgctcccttcccgccgcctcctccccgccctcctccctcctcgctcgcgggccgggcccggcatggtgcggcgtcgccgccgatggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgcgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgttagcccacaggcagagggcatcggggccatttggccggctctggtggccactgagctggctgactaagccccgtctgaccccagcccagacagcaaggtgtggaggctcctgtggtagtcctcccaagctgtgctgggaagcccggactgaccaaatcacccaatcccagttcttcctgcaaccactctgtggccagcctggcatcagtttaggccttggcttgatggaagtgggccagtcctggttgtctgaacccaggcagctggcaggagtggggtggttatgtttccatggttaccatgggtgtggatggcagtgtggggagggcaggtccagctctgtgggccctaccctcctgctgagctgcccctgctgcttaagtgcatgcattgagctgcctccagcctggtggcccagctattaccacacttggggtttaaatatccaggtgtgcccctccaagtcacaaagagatgtccttgtaatattcccttttaggtgagggttggtaaggggttggtatctcaggtctgaatcttcaccatctttctgattccgcaccctgcctacgccaggagaagttgaggggagcatgcttccctgcagctgaccgggtcacacaaaggcatgctggagtacccagcctatcaggtgcccctcttccaaaggcagcgtgccgagccagcaagaggaaggggtgctgtgaggcttgcccaggagcaagtgaggccggagaggagttcaggaacccttctccatacccacaatctgagcacgctaccaaatctcaaaatatcctaagactaacaaaggcagctgtgtctgagcccaacccttctaaacggtgacctttagtgccaacttcccctctaactggacagcctcttctgtcccaagtctccagagagaaatcaggcctgatgagggggaattcctggaacctggaccccagccttggtgggggagcctctggaatgcatggggcgggtcctagctgttagggacatttccaagctgttagttgctgtttaaaatagaaataaaattgaagactaaagacctaaaaaaaaaaaaaaaaaaaaaaaaaaaaaaaa

SEQ ID NO: 7 - protein, 885 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 8 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 9 - DNA, 4743 bp, Homo sapiens
gggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccaacaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctactttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 10 - DNA, 4716 bp, Homo sapiens
gggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccaacaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctactttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 11 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 12 - DNA, 3632 bp, Homo sapiens
actcactgcccgggccgcccggacagggagcttcgctggcgcgcttggccggcgacaggacaggttcgggacgtccatctgtccatccgtccggagagaaattacagatccgcagccccgggatggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcagtgtacctaatatataccaggacaccacaatttcttggtggaaagatgggaaggaattgcttggggcacatcatgcaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatccccctccgtgctaactgttccaggcctgacggagatggcggtcttcagttgtgaggcccacaatgacaaagggctgaccgtgtccaagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgagtaatggctcagtcatgatttttaacacctctgccttaccacatctgtaccaaatcaagcagctgcaagccctggctaattacagcattggtgtttcctgcatgaatgaaataggctggtctgcagtgagcccttggattctagccagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtgaggagaggtgcggggagacattccaaaaatcaagccaattcttctgctgtaggagaatccaattgtacctgatgtttttggtatttgtcttccttaccaagtgaactccatggccccaaagcaccagatgaatgttgttaagtaagctgtcattaaaaatacataatatatatttatttaaagagaaaaaatatgtgtatatcatggaaaaagacaaggatattttaataaaacattacttatttcatttcacttatcttgcatatcttaaaattaagcttcagctgctccttgatattaacatttgtacagagttgaagttgttttttcaagttcttttctttttcatgactattaaatgtaaaaatatttgtaaaatgaaatgccatatttgacttggcttctggtcttgatgtatttgataagaatgattcattcaatgtttaaagttgtataactgattaattttctgatatggcttcctaataaaatatgaataaggaagaaaaaa

SEQ ID NO: 13 - protein, 678 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 14 - protein, 405 aa, Homo sapiens
MDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 15 - protein, 379 aa, Homo sapiens
MFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 16 - DNA, 2521 bp, Homo sapiens
ccgagcgcttgaggtgccgcagccgccgccgccgccgccgccgcgatgtgaccttcagggccgccaggacgggatgaccggagcctccgccccgcggcgcccgcggctcgcctcggcctcccgggcgctctgaccgcgcgtccccggcccgccatggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaaaaaaaaaaaaaaaaaa

SEQ ID NO: 17 - DNA, 2188 bp, Homo sapiens
ttgattgaaaccagtaaatgcttctctttggggttggggttttagtttcaaatgcccccggggggttactttttacggccccgtgtcctgtagcaccgtcatttaaatggaacagcacagcgtgcaccgccgccccccacccctccaccaagcagggcccttcccagctctccacctgctgggctgaagtcagccttcccagccgggccttgatcagaagcgtgcaccaacaccccgggagctgcccggtcaggggaggagggcagggaaatggggccagggcgcgctggccccacagagtctggatgcgacctctgggtggtgccctggccagtccctgcagccgcctgccccagccccgtctgagatgccgctgtgctgcggttggccggtttttttttgcttgcagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaaaaaaaaaaaaaaaaaa

SEQ ID NO: 18 - DNA, 2523 bp, Homo sapiens
cacaccgacctgtcacaccggtgcctgtcacaccactgcctgtcacactgacttgtcaccggtgtctgtcacaccgacctgtcacactggtgcctgtcacactggtgcctgtcacaccgacctgtcacaccggtgcctgtcacaccgacctgtcacactgacctgtcacaccggtaggaatgcagtacccacatgtggacgtttctgggcagggcggctcttgtctttcctcttcagcctgggcctgtgcctgggggttgatgagagtgagcatttatttaaaaagcaaaaccacaggtggaaagagtcaccaggacagcttctcggagtcgcagacctgggatgcagccgtggggctcttgggtctgggctgcgacgttcagggcttccagccagccctcgccttgaggttctttgcctcgctgcctcatgtactcatgcagagggtgtcggacccctgcgagatgtccagctcaccctggctgcccacggtgggcagggcaggcctggctcagccccagcccctccatcttccaggggtgtcagctcacaccggctttggttctgtcccccttcgggcagcgtggagaaaccacagcccagaacagggaactttccaggacagccatcttcaaggcatccatatctatttcataatagtgtatactttttaatgattctctgtaatttttgtatgcttgaaatatttcataatttaaaaataaagggtcaagggaaatgagcagggaaggagatgacggggacccccgagaagccctgtgggaagcggctgctgcaagcccgcccttcacctgggagtcccagtggggcaggtgtgacagcctctggggtctcagcagctagaggcggggtggccactcccgaggcacaggagggacagtggacccgctgcgcggccggggcgtggggctcaggggagcaggagtgaaggccacatccccgaccggcgtggcccccgtccgtggcaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaaaaaaaaaaaaaaaaaa

SEQ ID NO: 19 - protein, 559 aa, Artificial Sequence
  Variant Gas6deltaGla protein
DQCTPNPCDKKGTHICQDLMGNFFCVCTDGWGGRLCDKDVNECVQKNGGCSQVCHNKPGSFQCACHSGFSLASDGQTCQDIDECTDSDTCGDARCKNLPGSYSCLCDEGYTYSSKEKTCQDVDECQQDRCEQTCVNSPGSYTCHCDGRGGLKLSPDMDTCEDILPCVPFSMAKSVKSLYLGRMFSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATP

SEQ ID NO: 20 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 21 - DNA, 4743 bp, Homo sapiens; identical to SEQ ID NO: 9

SEQ ID NO: 22 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 23 - DNA, 1841 bp, Homo sapiens
attctcctgcctcagcctcccgagtagctgggactacaggcgccagtgaccacgcccggctaattttttgtatttttagtagagacggggtttcacccttttagccaggatggtctcgatctcctgacttcgtgatctgcccgccttggcctcccaaagtgctaggattacaggtttgagccaccgcgcccggccctgtttcctttttgtttgttcccctgataccctgtatcaggaccaggagtcagtttggcggttatgtgtggggaagaagctgggaagtcaggggctgtttctgtggacagctttccctgtcctttggaaggcacagagctctcagctgcagggaactaacagagctctgaagccgttatatgtggtcttctctcatttccagcagagcaggctcatatgaatcaaccaactgggtgaaaagataagttgcaatctgagatttaagacttgatcagataccatctggtggagggtaccaaccagcctgtctgctcattttccttcaggctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgct

SEQ ID NO: 24 - DNA, 1493 bp, Homo sapiens
attctcctgcctcagcctcccgagtagctgggactacaggcgccagtgaccacgcccggctaattttttgtatttttagtagagacggggtttcacccttttagccaggatggtctcgatctcctgacttcgtgatctgcccgccttggcctcccaaagtgctaggattacaggctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgct

SEQ ID NO: 25 - DNA, 1359 bp, Homo sapiens
gttacccagcattgtgagtgacagagcctggatctgaacgctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgct

SEQ ID NO: 26 - protein, 142 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGEWTFACHLYE

SEQ ID NO: 27 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 28 - DNA, 2448 bp, Homo sapiens
agaacacttacaggatgtgtgtagtgtggcatgacagagaactttggtttcctttaatgtgactgtagacctggcagtgttactataagaatcactggcaatcagacacccgggtgtgctgagctagcactcagtgggggcggctactgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgttaaaaaaaaaaaaaaaaaa

SEQ ID NO: 29 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 30 - protein, 350 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 31 - DNA, 1374 bp, Homo sapiens
ataagaggttgggctttggatagatagacagactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaacttctctgtcaaaaa

SEQ ID NO: 32 - DNA, 1290 bp, Homo sapiens
ataagaggttgggctttggatagatagacagactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaacttctctgtcaaaaa

SEQ ID NO: 33 - RNA, 19 nt, Artificial Sequence
  siRNA against TIM-1 receptor
aaacucaacuguuccuaca

SEQ ID NO: 34 - RNA, 19 nt, Artificial Sequence
  siRNA against TIM-1 receptor
cggaaggacacacgcuaua

SEQ ID NO: 35 - RNA, 19 nt, Artificial Sequence
  siRNA against TIM-1 receptor
gcagaaacccacccuacga

SEQ ID NO: 36 - RNA, 19 nt, Artificial Sequence
  siRNA against TIM-1 receptor
ggucacgacuacuccaauu

SEQ ID NO: 37 - protein, 359 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD